(19) United States
(12) Patent Application Publication
(10) Pub. No.: US 2006/0041248 A1
(43) Pub. Date: Feb. 23, 2006

(54) Pharmaceutical compositions delivery system and methods

(76) Inventors: David L. Patton, Webster, NY (US); John P. Spoonhower, Webster, NY (US); David J. Nelson, Rochester, NY (US); John M. Pochan, Penfield, NY (US); Timothy Keefe, Rochester, NY (US); Kenneth J. Reed, Rochester, NY (US); Rajesh V. Mehta, Rochester, NY (US); Ramesh Jagannathan, Rochester, NY (US)

Correspondence Address: Pamela R. Crocker;Patent Legal Staff, Eastman Kodak Company, 343 State Street, Rochester, NY, 14650-2201, US

(21) Appl. No.: 10/923,937

(22) Filed: Aug. 23, 2004

(51) Int. Cl. A61K 9/22

(52) U.S. Cl. 604/890.1

(57) ABSTRACT

A method, system and applicator for the solvent-less delivery of a bio-active material to a receiver. The applicator includes a discharge device, a reservoir which holds a bioactive material, and a solvent at a supercritical fluid state for delivering the bioactive material through the discharge device to the receiver. A spacer may be positioned between the discharge device and the receiver. The receiver may have a plurality of different bio-active material to be applied to a subject, each located at a different location on the receiver.

### CROSS-REFERENCE TO RELATED APPLICATIONS

Reference is made to commonly assigned U.S. patent application Ser. No. 10/814,354, filed Mar. 31, 2004, entitled, “PROCESS FOR THE FORMATION OF PARTICULATE MATERIAL” by Rajesh Vinodria Mehta et al.

### FIELD OF THE INVENTION

This invention relates generally to the administration of compositions (such as pharmaceutical compositions or bioactive materials) for cutaneous administration, including transdermal and transmucuosal administration. In addition, this invention relates to the creation of orally administered patches, and wafers for the delivery of such compositions. In particular, this invention combines the technologies of pharmaceutical administration and solvent-less delivery systems.

### BACKGROUND OF THE INVENTION

Pharmaceutical compositions provide effective treatments for a variety of illnesses. Unfortunately, there are many obstacles to the administration of therapeutically effective doses of many medications. For example, some drugs (particularly peptide based drugs such as insulin) are partially or totally inactivated following oral ingestion, by the highly acidic environment of the stomach. Another problem is the “first pass” effect, which refers to the partial inactivation of orally ingested drugs in the liver, after they have been absorbed from the gastrointestinal system, but before they have exerted their full therapeutic effect. Even when these problems are overcome, patients often fail to take their medications at the proper prescribed intervals, or for the necessary period of time, to achieve an optimal therapeutic response.

Inhalation and intranasal administration has been used as alternative routes of drug delivery. Inhaled drugs can be absorbed directly through the mucous membranes and epithelium of the respiratory tract, thereby minimizing initial inactivation of bioactive substances by the liver. Inhalational delivery can also provide drugs directly to therapeutic sites of action (such as the lungs or the sinuses). This mode of administration has been particularly effective for the delivery of pulmonary drugs (such as asthma medications) and peptide based drugs (usually via intranasal administration), using metered dose inhalers (MDls). However, MDIs often require coordinating inspiration with actuation of the MDI, and some patients are not able to master this technique. Moreover, patients still often forget to take the medication at prescribed times, or for the necessary period of time to achieve clinical goals. Other patients inadvertently or inappropriately use medications, leading to hospitalizations, morbidity, and even death.

In an effort to overcome such problems, some drugs are administered by passive cutaneous routes, such as transdermal delivery of drugs from a patch applied to the skin. Examples of drugs that are routinely administered by this route are nitroglycerin, steroid hormones, and some analgesics (such as fentanyl). Transdermal administration avoids initial inactivation of drugs in the gastrointestinal tract, and provides continuous dosages usually over a relatively short period of time (such as a day), without requiring active participation by the patient. Continuous sustained administration provides better bioavailability of the drug, without peaks and troughs, and eliminates the problem of the patient forgetting to take multiple doses of the drug throughout the day. However, the patch must be changed regularly, usually each day, to provide a necessary drug concentration in the patch to establish the correct concentration gradient for delivery of the appropriate dose of the drug across the skin.

In addition to transdermal systemic delivery of drugs, topical delivery of drugs to the surface of the skin is also used for treating many skin conditions. For example, antibiotics are topically administered to the skin to treat infection, anesthetics to treat pain, retinoids to treat acne, and minoxidil to treat hair loss. These drugs must be repeatedly applied to the skin to achieve their effect, and much of the dosage may be lost by drainage of liquid from the application site, or being inadvertently wiped away. Moreover, excess drug is usually applied to the skin, which can lead to undesired toxic effects particularly if the drug is absorbed through the skin.

Pharmaceutical compositions can also include various agents that enhance or improve disease diagnosis. For example, in U.S. Pat. No. 6,592,847(B1), by Weissleder et al., an optical imaging probe and method is disclosed. This invention features an in-vivo optical imaging method comprising: (a) administering to a living animal or human an intramolecularly-quenched fluorescence probe comprising a fluorochrome attachment moiety and a plurality of near infrared fluorochromes covalently linked to the fluorochrome attachment moiety at fluorescence-quenching interaction-permissive positions. These positions are separable by enzymatic cleavage at fluorescence activation sites, which enzymatic cleavage occurs preferentially in a target tissue; (b) allowing time for enzymes in the target tissue to activate the probe by enzymatic cleavage at fluorescence activation sites, if the target tissue is present; (c) illuminating the target tissue with near infrared light of a wavelength absorbable by the fluorochromes; and (d) detecting fluorescence emitted by the fluorochromes. The delivery of such optical imaging probes can radically improve disease diagnosis.

Devices and methods are disclosed herein for improving the cutaneous delivery of pharmaceutical compositions, by using solvent-less applicators for transdermal and other cutaneous delivery of such compositions, as well as orally administered delivery. Systems for administrating bioactive materials in this fashion are also described. “Receiver” for the purposes of this description can comprise all of the above mentioned delivery methods direct to skin, patches etc.

Technologies that deposit a marking material such as a toner particle onto a receiver using gaseous propellants are known. For example, Peeters et al., in U.S. Pat. No. 6,116,718, disclose a print head for use in a marking apparatus in which a propellant gas is passed through a channel, the functional material is introduced controllably into the propellant stream to form a ballistic aerosol for propelling non-colloidal, solid or semi-solid particulate or a liquid, toward a receiver with sufficient kinetic energy to fuse the marking material to the receiver. There is a problem with this technology in that the functional material and propellant stream are two different entities and the propellant is used to impart kinetic energy to the functional material. This can cause functional material agglomeration leading to nozzle obstruction and poor control over functional material deposition. Another problem with this technology is that when the functional material is added into the propellant stream in the channel it forms a non-colloidal ballistic aerosol prior to exiting the print head. This non-colloidal ballistic aerosol, which is a combination of the functional material and the propellant, is not thermodynamically stable. As such, the functional material is prone to settling in the propellant stream that, in turn, can cause functional material agglomeration leading to nozzle obstruction and poor control over functional material deposition.

Additionally, there is a need for a technology capable of controlled functional material deposition within a receiver or within a predetermined layer of a receiver. There is also a need for a technology that permits functional material deposition of ultra-small (nano-scale) particles.

Jagannathan et al. in U.S. Pat. No. 6,471,327(B2), incorporated by reference, disclose a method and apparatus for delivery of a focused beam of a thermodynamically stable/metastable mixture of functional material and a dense gas. The apparatus disclosed in Jagannathan et al. can be directly implemented in delivery of bioactive materials to a receiver.

U.S. 2003/0107614 A1, U.S. 2003/0227502 A1, U.S. 2003/0132993 A1, and U.S. 2003/0227499 A1, incorporated by reference, define various additions and further concepts for providing an apparatus and method for printing with a thermodynamically stable mixture of a fluid and marking material. The teachings of the above applications on print head design, the use of multiple marking materials, cleaning and calibration, can be applied to the delivery of bioactive materials.

An object of the present invention is to provide a technology that permits high speed, accurate, and precise deposition of a solvent free bioactive material on a receiver.

Another object of the present invention is to provide a technology capable of controlled bioactive material deposition within a receiver or within a predetermined layer of a receiver.

According to a feature of the present invention, a method of delivering a bioactive material to a receiver includes in order, providing a mixture of a fluid having a solvent and a bioactive material; causing the bioactive material to become free of the solvent; causing the bioactive material to contact a receiver, and penetrate said receiver to a predetermined layer.

According to another feature of the present invention, an apparatus for delivering a bioactive material to a receiver includes a pressurized source of solvent in a thermodynamically stable mixture with a bioactive material, the solvent being in a liquid state within the pressurized source. A discharge device having an inlet and an outlet, the discharge device being connected to the pressurized source at the inlet, the thermodynamically stable mixture being ejected from the outlet, the solvent being in a gaseous state at a location beyond the outlet of the discharge device.

According to another feature of the present invention, a method of delivering a bioactive material to a receiver includes providing a source of a thermodynamically stable mixture of a solvent in a liquid state and a bioactive material; providing a discharge device having a nozzle in fluid communication with the source of the thermodynamically stable mixture; positioning a receiver at a predetermined distance from the nozzle; ejecting the thermodynamically stable mixture from the nozzle, the solvent changing from the liquid state to a gaseous state; and depositing the solvent free bioactive material on the receiver.

### SUMMARY OF THE INVENTION

The present invention is directed to overcoming one or more of the problems set forth above. Briefly summarized, according to one aspect of the present invention, there is provided a system for solvent-less delivery of a bioactive material to a receiver, comprising: a discharge device, a reservoir which holds a bioactive material, and a solvent at a supercritical fluid state for delivering the bioactive material through the discharge device to the receiver.

According to another aspect of the present invention there is provided a system for solvent-less delivery of a bioactive material to a receiver, comprising: a discharge device, a spacer positioned between the discharge device and the receiver, a reservoir which holds a bioactive material, and a solvent at a supercritical fluid state for delivering the bioactive material through the discharge device to the receiver.

According to yet another aspect of the present invention there is provided a kit for solvent-less delivery of a bioactive material to a receiver, the kit comprising:
an applicator having a discharge device, a reservoir which holds a bioactive material, and a solvent at a supercritical fluid state for delivering the bioactive material through the discharge device; and
a receiver for receiving the bioactive material.

In accordance with still another aspect of the present invention there is provided a method for solvent-less delivery of a bioactive material to a receiver, comprising:
providing a discharge device, a reservoir which holds a bioactive material, and a solvent at a supercritical fluid state; and
delivering the bioactive material through the discharge device to the receiver.

In accordance with another aspect of the present invention there is provided a receiver for applying a bioactive material to a subject, the receiver having a plurality of different bio-active material to be applied to a subject, each located at a different location on the receiver.

In accordance with yet still another aspect of the present invention there is provided a receiver for applying a bioactive material to a subject, the receiver having a plurality of different bio-active material to be applied to a subject, each located at a different depth on the receiver.

These and other aspects, objects, features and advantages of the present invention will be more clearly understood and appreciated from a review of the following detailed description of the preferred embodiments and appended claims and by reference to the accompanying drawings.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIG. 1A is a schematic view of an embodiment made in accordance with the present invention;

FIGS. 1B-1G are schematic views of alternative embodiments made in accordance with the present invention;

FIG. 1H is a schematic view of another embodiment made in accordance with the present invention;

FIG. 2A is a schematic of the discharge device of FIG. 1 made in accordance with the present invention;

FIGS. 2B-2J are cross sectional views of various different discharge nozzles of the discharge device shown in FIG. 2A;

FIGS. 3A-3D are schematic diagrams showing the operation of a delivery system made in accordance with the present invention;

FIGS. 4A-4K are cross-sectional views of a reservoir for use in the present invention having various different temperature and pressure control mechanisms for use in the present invention;

FIGS. 5A-5D are schematic views of a beam control device for controlling the ejected formulation and a spacer-shield used in the discharge device made in accordance with the present invention;

FIG. 6 is a cross-sectional view of a portion of another embodiment of the spacer-shield for use with the discharge device;

FIG. 7 is a perspective view of a delivery system made in accordance with the present invention for printing onto a receiver where the receiver is the surface of the skin;

FIG. 8A is a cross-sectional view of a portion of the receiver made in accordance wherein the receiver comprises a transdermal patch;

FIG. 8B is a perspective and partial schematic view of the receiver made in accordance with the present invention wherein the transdermal patch of FIG. 8A is placed on the surface of the skin;

FIG. 9A is a cross-sectional view of a portion of another embodiment of the receiver made in accordance with the present invention wherein the receiver comprises a multi-layer time-release material:

FIG. 9B is a cross-sectional view of a portion of yet another embodiment of a receiver made in accordance with the present invention that comprises a single layer time-release material;

FIG. 10 is a schematic view of a delivery system made in accordance with the present invention being used for printing onto a receiver where the receiver is a transdermal patch;

FIG. 11 is a schematic view similar to FIG. 10 wherein the receiver is a freeze-dried gelatinous oral material;

FIG. 12 is a schematic view similar to FIG. 10 wherein the receiver is a multi-layer time-release material;

FIG. 13 is a schematic view of a multiple delivery system made in accordance with the present invention;

FIG. 14 is a schematic view of another delivery system made in accordance with the present invention;

FIG. 15 is a schematic view of yet another embodiment of the multiple delivery system made in accordance with the present invention.

To facilitate understanding, identical reference numerals have been used, where possible, to designate identical elements that are common to the figures.

### DETAILED DESCRIPTION OF THE INVENTION

Unless otherwise noted, technical terms are used according to conventional usage. Definitions of common terms in pharmacology may be found in Remington: The Science and Practice of Pharmacy, 19th Edition, published by Mack Publishing Company, 1995 (ISBN 0-912734-04-3). Transdermal delivery is discussed in particular at page 743 and pages 1577-1584.

The singular forms “a,” “an,” and “the” refer to one or more than one, unless the context clearly dictates otherwise. The term “comprising” means “including.”

An “array” refers to a predetermined pattern, which can be either regular or irregular. Examples of arrays are linear distributions or two-dimensional matrices.

A “bioactive” material, composition, substance or agent is a composition which affects a biological function of a subject to which it is administered. An example of a bioactive material used to create a composition is a pharmaceutical substance, such as a drug, which is given to a subject to alter a physiological condition of the subject, such as a disease. Bioactive materials, compositions and agents also include other biomolecules, such as proteins and nucleic acids, or liposomes and other carrier vehicles that contains bioactive materials. A “bioactive” composition can also include various agents that enhance or improve disease diagnosis. For example, in U.S. Pat. No. 6,592,847(B1), by Weissleder et al., an optical imaging probe and method is disclosed. This invention features an in-vivo optical imaging method comprising: (a) administering to a living animal or human an intramolecularly-quenched fluorescence probe comprising a fluorochrome attachment moiety and a plurality of near infrared fluorochromes covalently linked to the fluorochrome attachment moiety at fluorescence-quenching interaction-permissive positions. These positions are separable by enzymatic cleavage at fluorescence activation sites, which enzymatic cleavage occurs preferentially in a target tissue; (b) allowing time for enzymes in the target tissue to activate the probe by enzymatic cleavage at fluorescence activation sites, if the target tissue is present; (c) illuminating the target tissue with near infrared light of a wavelength absorbable by the fluorochromes; and (d) detecting fluorescence emitted by the fluorochromes. The delivery of such optical imaging probes can radically improve disease diagnosis.

“Cutaneous” refers to the skin, and “cutaneous delivery” means application to the skin. This form of delivery can include either delivery to the surface of the skin to provide a local or topical effect, or transdermal delivery. The following terms are intended to be defined as indicated below. The term “transdermal”, delivery captures both transdermal (or “percutaneous”) and transmucosal administration, i.e., delivery by passage of a bioactive material through the skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Aspects of the invention which are described herein in the context of “transdermal” delivery, unless otherwise specified, are meant to apply to both transdermal and transmucosal delivery. That is, the compositions, systems, and methods of the invention, unless explicitly stated otherwise, should be presumed to be equally applicable to transdermal and transmucosal modes of delivery.

The present disclosure is directed to solvent-less delivery systems that are based on the use of supercritical fluids. Supercritical fluids have unique properties, since they combine liquid-like solvent power with gas-like transport properties. They have a large compressibility compared to ideal gases. Therefore, a small change in temperature or pressure near the critical values will result in large changes in the fluid's density and hence its solvent power. These characteristics can be utilized to provide highly controllable solvent properties. Carbon dioxide is the most widely used supercritical fluid, due to the favorable critical parameters (Tc=31.1° C., Pc=73.8 bar), cost and non-toxicity.

This invention is directed more specifically at a compressed fluid based device such as is disclosed in U.S. Ser. No. 10/814,354 filed Mar. 31, 2004 entitled PROCESS FOR THE FORMATION OF PARTICULATE MATERIAL by Rajesh Vinodrai Mehta et al., and U.S. Pat. No. 6,752,484, both of which are incorporated by reference herein in their entirety and are explained in detail below. A significant feature of this invention is that precipitated particles of sizes less than 100 nanometers can be produced free of high levels of non-uniform large particles. The delivery system includes a container for holding the bioactive material and delivering it to a dispenser nozzle, or an array of dispenser nozzles. The spray from the delivery system is self-energized obviating the need for additional energy sources to propel the bioactive material from the delivery system toward a cutaneous target.

The medication dispensers disclosed herein may be similar to liquid dispensers known as solvent-less print heads used in solvent-less printing mechanisms, such as printers, plotters, facsimile machines and the like, some of which are described for example in Durbeck and Sherr, Output Hardcopy Devices, Academic Press Inc., 1987 (ISBN 0-12-225040-0), particularly in chapter 13, pages 311-370. These technologies have in common the extraction of small quantities of a fluid from a reservoir, which are converted into fine droplets, and transported through the air to a target medium by appropriate application of physical forces. This technology has been implemented in a variety of ways, but one of the common approaches has been thermal solvent-less technology, in which liquids are heated using resistors to form drops and propel them from a chamber through an orifice toward a target. Another approach is piezoelectric solvent-less technology, in which movement of a piezoelectric transducer changes a chamber volume to generate the drop. An additional approach is known as silicon electrostatic actuator (“SEA”) solvent-less technology, such as that disclosed in U.S. Pat. No. 5,739,831 to Nakamura (assigned to Seiko Epson Corporation).

In striving to duplicate the quality of photographic film images, the solvent-less printing industry has focused on decreasing the size of ink droplets ejected from the nozzles, as well as accurately placing these droplets on the print media. For instance, some of the more recent solvent-less print cartridges are able to deliver droplets of a size on the order of 0.5-6 Pico liters, although larger droplets can also be generated, for example droplets of 10, 50, 100 or more Pico liters. The resolution within which currently commercially available solvent-less printing mechanisms may place ink droplets on a page is on the order of 1200-4800 dots per inch (known in the industry as a “dpi” rating). Thus, while striving to achieve photographic print quality, solvent-less printing technology has become very adept at accurately metering and dispensing fluids. The ability to dispense very small and accurate amounts of fluids (including liquids and powders) is taken advantage of in constructing the transdermal cutaneous application systems illustrated herein.

While these solvent-less print heads may be used in the cutaneous application systems illustrated here, rather than using a printing analogy, the print head will instead be referred to in a more general nature as a “discharge device”, “delivery system” or “applicator.”

The bioactive material may be any flowable fluid (for example a liquid, gel or powder), although liquids are particularly useful in the delivery system. In some embodiments, at least one of the container modules may contain a bioactive material in powder or other dry form. The powder or other material is dispensed from the container, and may be combined with a liquid (such as a penetration enhancer) en route to the cutaneous delivery site.

In certain embodiments, the delivery system includes the bioactive material in the container. Examples of bioactive materials that can be included in the container include pharmaceutical compositions that are capable of transdermal delivery. As used herein, the terms “bio active material” and/or “particles of a bioactive material” intend any compound or composition of matter which, when administered to an organism (human or nonhuman animal) induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. More particularly, the term “bioactive material” includes compounds or compositions for use in all of the major therapeutic areas including, but not limited to, anti-infectives such as antibiotics and antiviral agents; analgesics and analgesic combinations; local and general anesthetics; anorexics; anti-arthritics; anti-asthmatic agents; anticonvulsants; antidepressants; antihistamines; anti-inflammatory agents; antinauseates; anti-migraine agents; antineoplastics; antipruritics; antipsychotics; antipyretics; antispasmodics; cardiovascular preparations (including calcium channel blockers, beta-blockers, beta-agonists and antiarrythmics); anti-hypertensives; diuretics; vasodilators; central nervous system stimulants; cough and cold preparations; decongestants; diagnostics; hormones; bone growth stimulants and bone resorption inhibitors; immunosuppressives; muscle relaxants; psycho stimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including double- and single-stranded molecules and supercoiled or condensed molecules, gene constructs, expression vectors, plasmids, antisense molecules and the like). Particles of a bioactive material, alone or in combination with other drugs or agents, are typically prepared as pharmaceutical compositions which can contain one or more added materials such as carriers, vehicles, and/or excipients. “Carriers,” “vehicles” and “excipients” generally refer to substantially inert materials which are nontoxic and do not interact with other components of the composition in a deleterious manner. These materials can be used to increase the amount of solids in particulate pharmaceutical compositions. Examples of suitable carriers include silicone, gelatin, waxes, and like materials. Examples of normally employed “excipients,” include pharmaceutical grades of dextrose, sucrose, lactose, trehalose, mannitol, sorbitol, inositol, dextran, starch, cellulose, sodium or calcium phosphates, calcium sulfate, citric acid, tartaric acid, glycine, high molecular weight polyethylene glycols (PEG), erodible polymers (such as polylactic acid, polyglycolic acid, and copolymers thereof), and combinations thereof. In addition, it may be desirable to include a charged lipid and/or detergent in the pharmaceutical compositions. Such materials can be used as stabilizers, anti-oxidants, or used to reduce the possibility of local irritation at the site of administration. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, e.g., TWEEN® surfactants (Atlas Chemical Industries, Wilmington, Del.), polyoxyethylene ethers, e.g., Brij, pharmaceutically acceptable fatty acid esters, e.g., lauryl sulfate and salts thereof (SDS), and like materials.

A delivery system made in accordance with the present invention may also include a controller for manually or automatically dispensing the bioactive material from the delivery system at selected times. The controller may take the form of an actuator that is manually depressed to activate the delivery system and dispense the agent. Alternatively, the controller may be a microprocessor, which is programmed to dispense the bioactive material at predetermined intervals, for example several times a day, as needed, directly on to the skin or on to a patch to be applied to a subject.

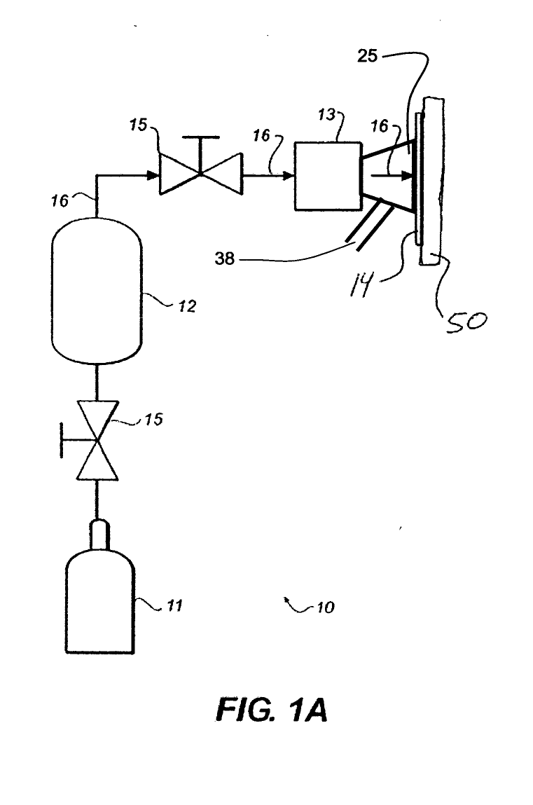

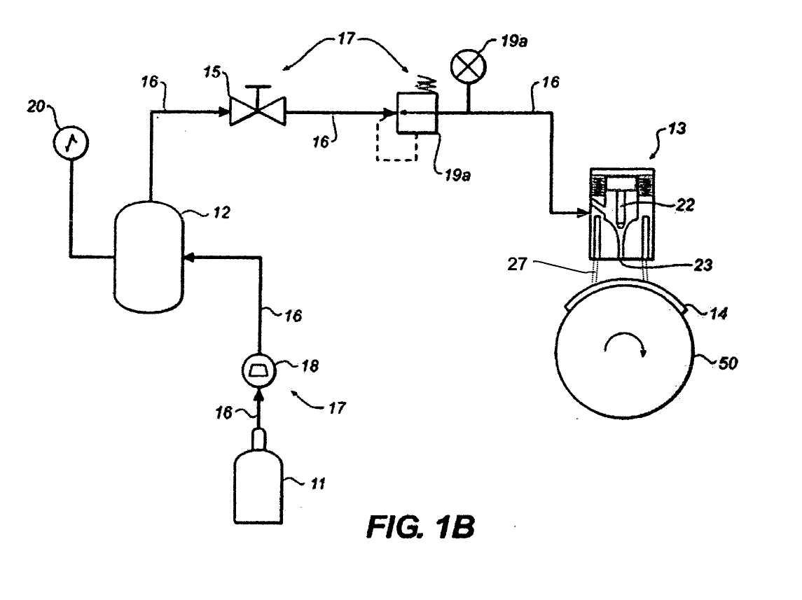

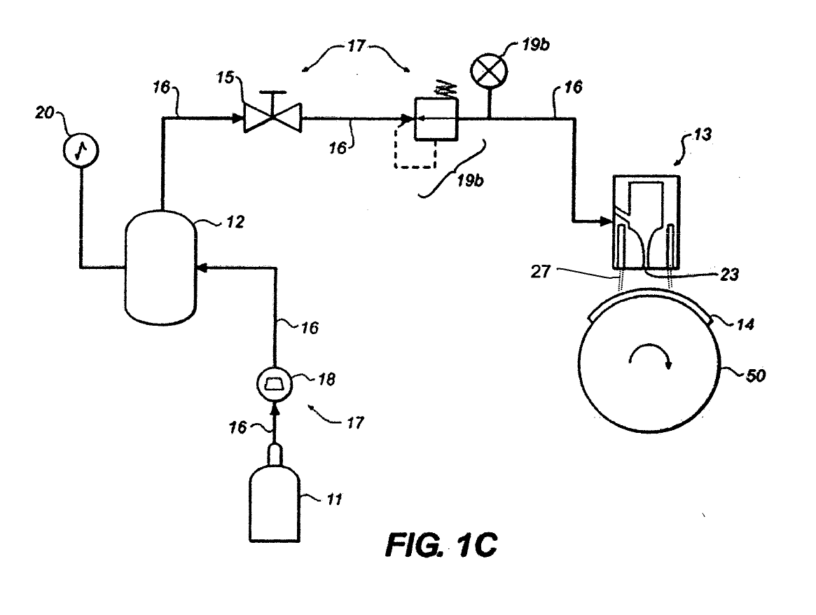

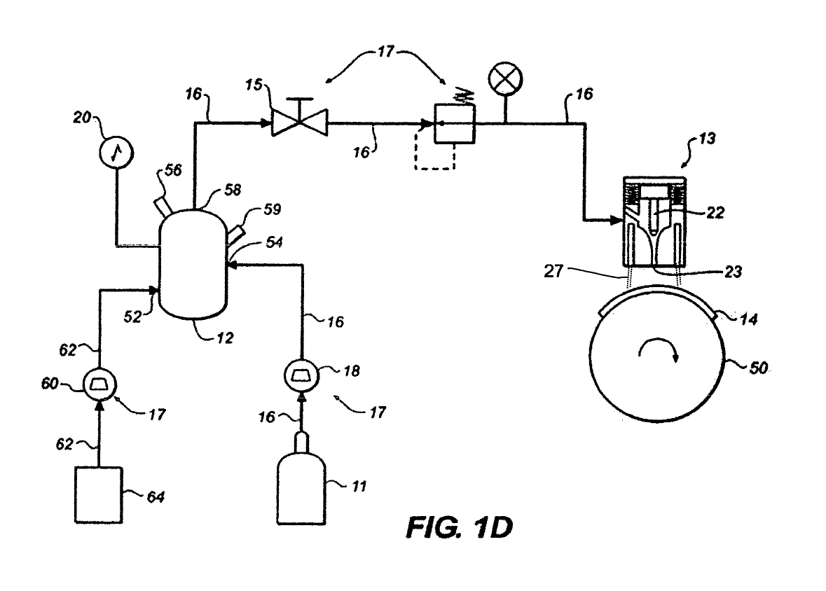

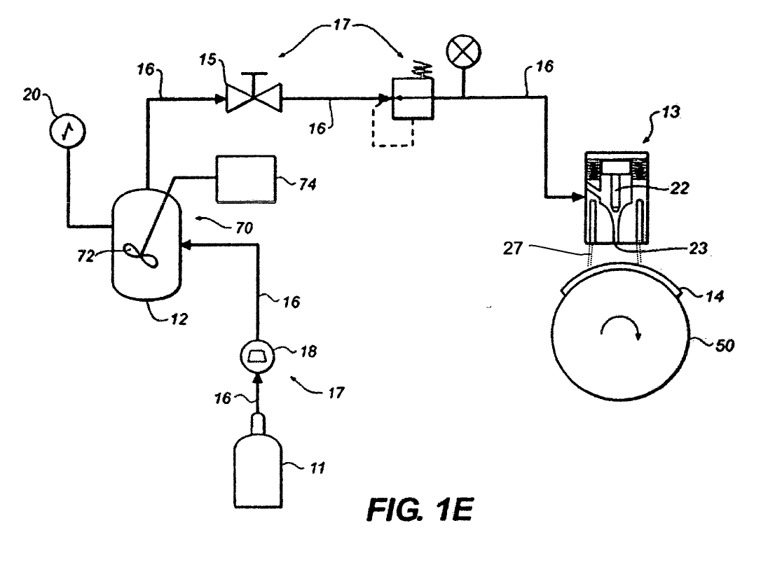

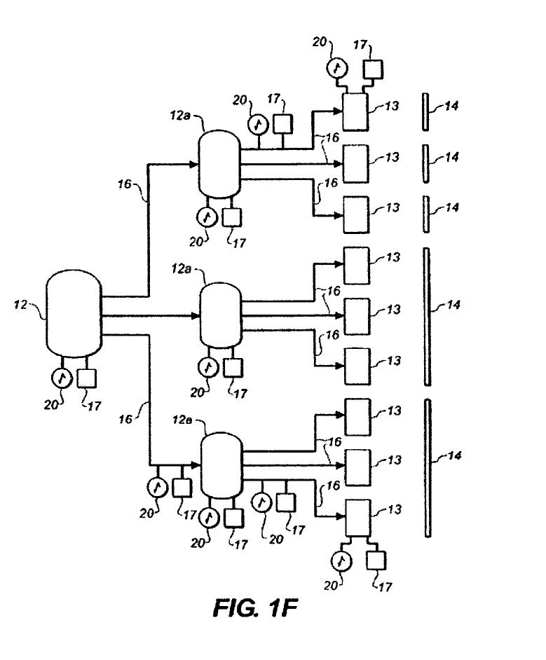

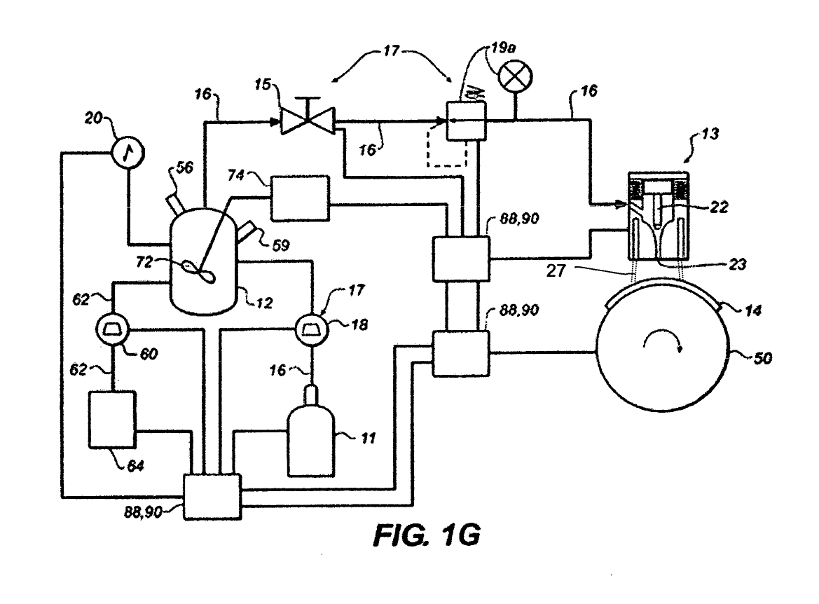

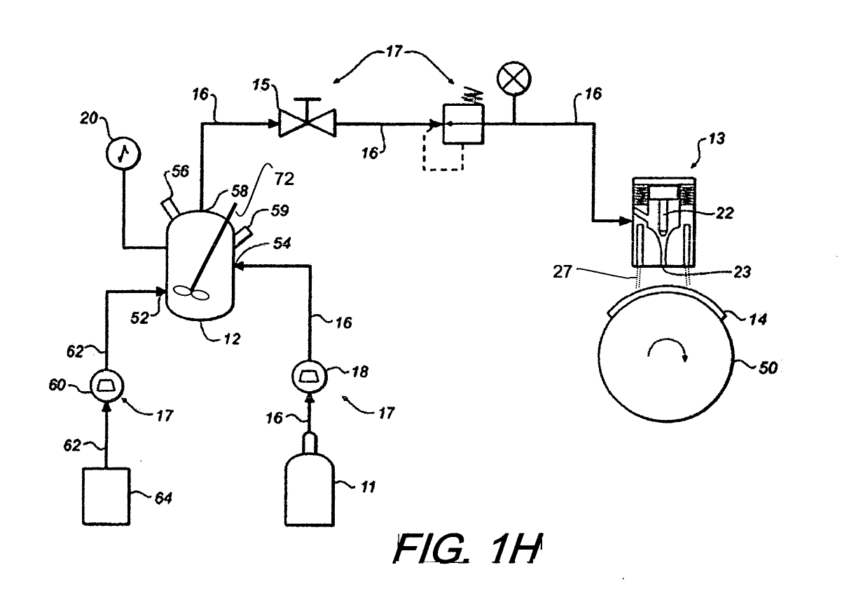

Referring to FIG. 1A, a delivery system 10 made in accordance with the present invention has components, 11, 12, and 13 that take chosen solvent and/or dispersant materials to a compressed liquid and/or supercritical fluid state, make a solution and/or dispersion of an appropriate bioactive material or combination of bioactive materials in the chosen compressed liquid and/or supercritical fluid, and deliver the bioactive materials as a collimated and/or focused beam onto a receiver 14 in a controlled manner. Bioactive materials can be any material that needs to be delivered to a receiver, for example a transdermal patch, a freeze-dried gelatinous oral patch and multi-layer time-release material (an oral time release system)

In this context, the chosen materials taken to a compressed liquid and/or supercritical fluid state are gases at ambient pressure and temperature. Ambient conditions are preferably defined as temperature in the range from −100 to +100° C., and pressure in the range from 1×10⁻⁸-00 atm for this application.

In FIG. 1A, a schematic illustration of the delivery system 10 is shown. The delivery system 10 has a compressed liquid/supercritical fluid source 11, a formulation reservoir 12, and a discharge device 13 connected in fluid communication along a delivery path 16. The delivery system 10 can also include a valve or valves 15 positioned along the delivery path 16 in order to control flow of the compressed liquid/supercritical fluid and a spacer-shield 25 for spacing of the discharge device from the receiver 14. To dissipate the gas portion of the super critical fluid, a gas release port 38 is connected to the space-shield. In another embodiment the space shield may be made of a porous material which would allow the gas portion of the super critical fluid to escape.

A compressed liquid/supercritical fluid carrier, contained in the compressed liquid/supercritical fluid source 11, is any material that dissolves/solubilizes/disperses a bioactive material. The compressed liquid/supercritical fluid source 11 delivers the compressed liquid/supercritical fluid carrier at predetermined conditions of pressure, temperature, and flow rate as a supercritical fluid, or a compressed liquid. Materials that are above their critical point, defined by a critical temperature and a critical pressure, are known as supercritical fluids. The critical temperature and critical pressure typically define a thermodynamic state in which a fluid or a material becomes supercritical and exhibits gas-like and liquid-like properties. Materials that are at sufficiently high temperatures and pressures below their critical point are known as compressed liquids. Materials in their supercritical fluid and/or compressed liquid state that exist as gases at ambient conditions find application here because of their unique ability to solubilize and/or disperse bioactive materials of interest in the compressed liquid or supercritical state.

Fluid carriers include, but are not limited to, carbon dioxide, nitrous oxide, ammonia, xenon, ethane, ethylene, propane, propylene, butane, isobutane, chlorotrifluoromethane, monofluoromethane, sulphur hexafluoride and mixtures thereof. Due to its characteristics, e.g. low cost, wide availability, etc., carbon dioxide is generally preferred in many applications.

The formulation reservoir 12 is utilized to dissolve and/or disperse bioactive materials in compressed liquids or supercritical fluids with or without dispersants and/or surfactants, at desired formulation conditions of temperature, pressure, volume, and concentration. The combination of bioactive material and compressed liquid/supercritical fluid is typically referred to as a mixture, formulation, etc.

The formulation reservoir 12 can be made out of any suitable materials that can safely operate at the formulation conditions. An operating range from 0.001 atmosphere (1.013×10² Pa) to 1000 atmospheres (1.013×10⁸ Pa) in pressure and from −25 degrees Centigrade to 1000 degrees Centigrade is generally preferred. Typically, the preferred materials include various grades of high pressure stainless steel. However, it is possible to use other materials if the specific deposition or etching application dictates less extreme conditions of temperature and/or pressure.

The formulation reservoir 12 should be precisely controlled with respect to the operating conditions (pressure, temperature, and volume). The solubility/dispersibility of bioactive materials depends upon the conditions within the formulation reservoir 12. As such, small changes in the operating conditions within the formulation reservoir 12 can have undesired effects on bioactive material solubility/dispensability.

Additionally, any suitable surfactant and/or dispersant material that is capable of solubilizing/dispersing the bioactive materials in the compressed liquid/supercritical fluid for a specific application can be incorporated into the mixture of bioactive material and compressed liquid/supercritical fluid. Such materials include, but are not limited to, fluorinated polymers such as perfluoropolyether, siloxane compounds, etc.

Referring to FIGS. 1B-1D, alternative embodiments of the invention shown in FIG. 1A are described. In each of these embodiments, individual components are in fluid communication, as is appropriate, along the delivery path 16.

Referring to FIGS. 1B and 1C, a pressure control mechanism 17 is positioned along the delivery path 16. The pressure control mechanism 17 is used to create and maintain a desired pressure required for a particular application. The pressure control mechanism 17 can include a pump 18, a valve(s) 15, and a pressure regulator 19a, as shown in FIG. 1B. Alternatively, the pressure control mechanism 17 can include a pump 18, a valve(s) 15, and a multi-stage pressure regulator 19b, as shown in FIG. 1C. Additionally, the pressure control mechanism 17 can include alternative combinations of pressure controlling devices, etc. For example, the pressure control mechanism 17 can include additional valve(s) 15, actuators to regulate fluid/formulation flow, variable volume devices to change system operating pressure, etc., appropriately positioned along the delivery path 16. Typically, the pump 18 is positioned along the delivery path 16 between the fluid source 11 and the formulation reservoir 12. The pump 18 can be a high pressure pump that increases and maintains system operating pressure, etc. The pressure control mechanism 17 can also include any number of sensor and/or monitoring devices, gauges, etc., for monitoring the pressure of the delivery system 10.

A temperature control mechanism 20 is positioned along delivery path 16 in order to create and maintain a desired temperature for a particular application. The temperature control mechanism 20 is preferably positioned at the formulation reservoir 12. The temperature control mechanism 20 can include a heater, a heater including electrical wires, a water jacket, a refrigeration coil, a combination of temperature controlling devices, etc. The temperature control mechanism 20 can also include any number of monitoring devices, gauges, etc., for monitoring the temperature of the delivery system 10.

The discharge device 13 includes a nozzle 23 positioned to provide direct delivery of the formulation towards the receiver 14. The discharge device 13 can also include a shutter 22 to regulate the flow of the supercritical fluid/compressed liquid and bioactive material mixture or formulation. The shutter 22 regulates flow of the formulation in a predetermined manner (i.e. on/off or partial opening operation at desired frequency, etc.). The shutter 22 can be manually, mechanically, pneumatically, electrically or electronically actuated. Alternatively, the discharge device 13 does not have to include the shutter 22 (shown in FIG. 1C). As the mixture is under higher pressure, as compared to ambient conditions, in the delivery system 10, the mixture will naturally move toward the region of lower pressure, the area of ambient conditions. In this sense, the delivery system 10 is said to be self-energized. The discharge device 13 can also include a spacer-shield 25 shown in FIGS. 1A-E, 5D, and 6, which is used to collect extraneous particles 49.

The receiver 14 can be positioned on a media conveyance mechanism 50 that is used to control the movement of the receiver during the operation of the delivery system 10. The media conveyance mechanism 50 can be a drum, an x, y, z translator, any other known media conveyance mechanism, etc.

Referring to FIG. 1D, the formulation reservoir 12 can be a pressurized vessel having appropriate inlet ports 52, 54, 56 and outlet ports 58. Inlet ports 52, 54, 56 can be used as an inlet port for bioactive material 64 and an inlet port for compressed liquid or supercritical fluid 11. Alternatively, inlet port 56 can be used to manually add bioactive material to the formulation reservoir 12. Outlet port 58 can be used as an outlet for the mixture of bioactive material and compressed/supercritical fluid.

When automated delivery of the bioactive material is desired, a pump 60 is positioned along a bioactive material delivery path 62 between a source of bioactive material 64 and the formulation reservoir 12. The pump 60 pumps a desired amount of bioactive material through inlet port 52 into the formulation reservoir 12. The formulation reservoir 12 can also include additional inlet/outlet ports 59 for inserting or removing small quantities of bioactive material or bioactive material and compressed liquid/supercritical fluid mixtures.

Referring to FIG. 1E, the formulation reservoir 12 can include a mixing device 70 used to create the mixture of bioactive material and compressed liquid/supercritical fluid. Although typical, a mixing device 70 is not always necessary to make the mixture of the bioactive material and compressed/supercritical fluid depending on the type of bioactive material and the type of compressed liquid/supercritical fluid. The mixing device 70 can include a mixing element 72 connected to a power/control source 74 to ensure that the bioactive material disperses into or forms a solution with the compressed liquid or supercritical fluid. The mixing element 72 can be an acoustic, a mechanical, and/or an electromagnetic element.

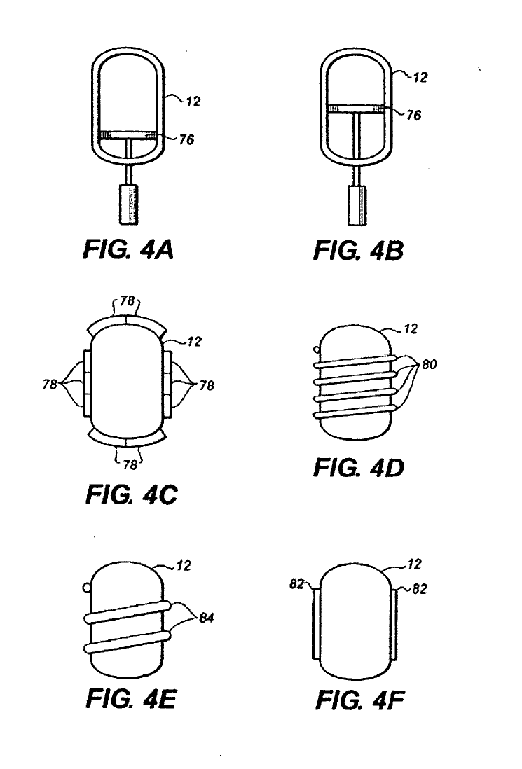

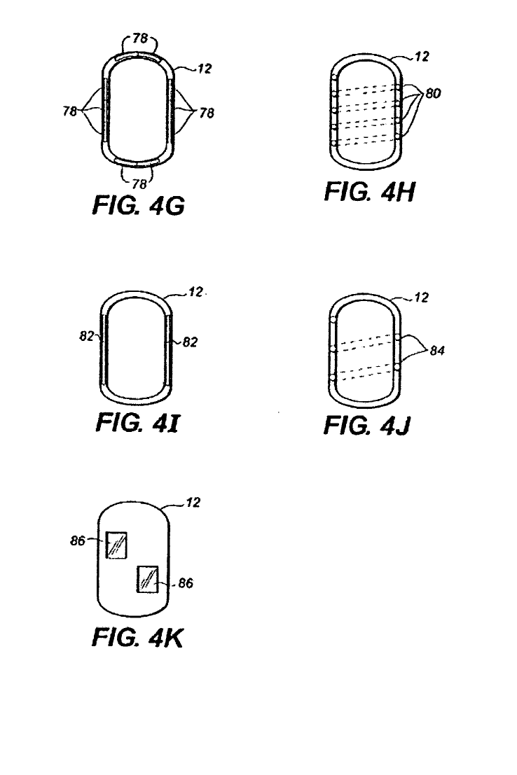

Referring to FIGS. 1D, 1E, and FIGS. 4A-4J, the formulation reservoir 12 can also include suitable temperature control mechanisms 20 and pressure control mechanisms 17 with adequate gauging instruments to detect and monitor the temperature and pressure conditions within the reservoir, as described above. For example, the formulation reservoir 12 can include a moveable piston device 76, etc., to control and maintain pressure. The formulation reservoir 12 can also be equipped to provide accurate control over temperature within the reservoir. For example, the formulation reservoir 12 can include electrical heating/cooling zones 78, using electrical wires 80, electrical tapes, water jackets 82, other heating/cooling fluid jackets, refrigeration coils 84, etc., to control and maintain temperature. The temperature control mechanisms 20 can be positioned within the formulation reservoir 12 or positioned outside the formulation reservoir. Additionally, the temperature control mechanisms 20 can be positioned over a portion of the formulation reservoir 12, throughout the formulation reservoir 12, or over the entire area of the formulation reservoir 12.

Referring to FIG. 4K, the formulation reservoir 12 can also include any number of suitable high-pressure windows 86 for manual viewing or digital viewing using an appropriate fiber optics or camera set-up. The windows 86 are typically made of sapphire or quartz or other suitable materials that permit the passage of the appropriate frequencies of radiation for viewing/detection/analysis of reservoir contents (using visible, infrared, X-ray etc. viewing/detection/analysis techniques), etc.

The formulation reservoir 12 is made of appropriate materials of construction in order to withstand high pressures of the order of 10,000 psi or greater. Typically, stainless steel is the preferred material of construction although other high pressure metals, metal alloys, and/or metal composites can be used.

Referring to FIG. 1F, in an alternative arrangement, the thermodynamically stable/metastable mixture of bioactive material and compressed liquid/supercritical fluid can be prepared in one formulation reservoir 12 and then transported to one or more additional formulation reservoirs 12a. For example, a single large formulation reservoir 12 can be suitably connected to one or more subsidiary high pressure vessels 12a that maintain the bioactive material and compressed liquid/supercritical fluid mixture at controlled temperature and pressure conditions with each subsidiary high pressure vessel 12a feeding one or more discharge devices 13. Either or both reservoirs 12 and 12a can be equipped with the temperature control mechanism 20 and/or pressure control mechanisms 17. The discharge devices 13 can direct the mixture towards a single receiver 14 or a plurality of receivers 14.

Referring to FIG. 1G, the delivery system 10 can include ports for the injection of suitable bioactive material, view cells, and suitable analytical equipment such as Fourier Transform Infrared Spectroscopy, Light Scattering, Ultraviolet or Visible Spectroscopy, etc. to permit monitoring of the delivery system 13 and the components of the delivery system. Additionally, the delivery system 10 can include any number of control devices 88 and/or microprocessors 90, etc., used to control the delivery system 10.

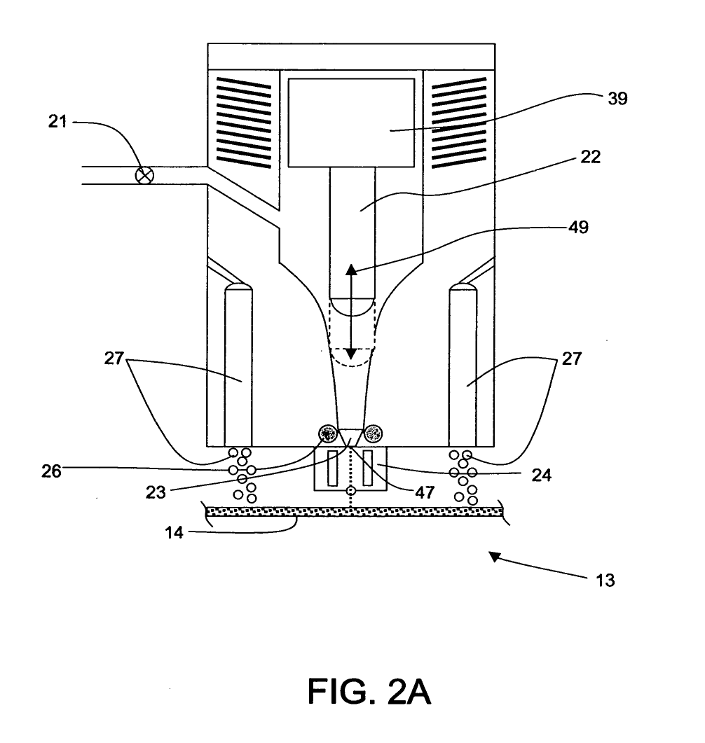

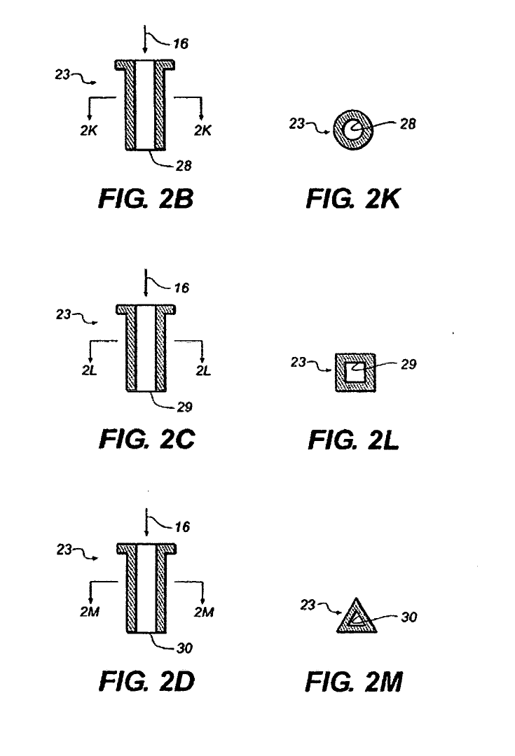

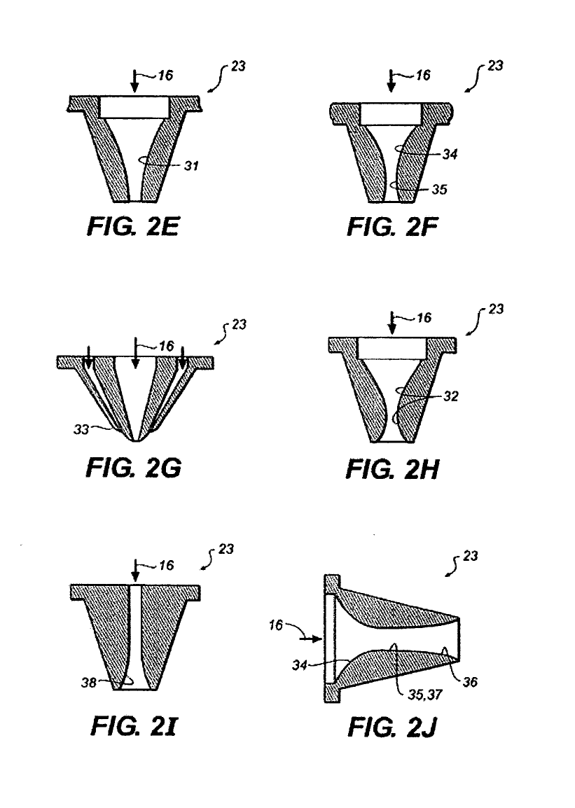

Referring to FIG. 2A, there is illustrated in greater detail a discharge device 13 made in accordance with the present invention. The discharge device 13 may include a control valve 21 that can be manually or automatically actuated to regulate the flow of the supercritical fluid or compressed liquid formulation. The discharge device 13 includes a shutter device 22 which can also be a programmable valve. The shutter device 22 is capable of being controlled to turn off the flow and/or turn on the flow via a solenoid 39 as shown by the arrow 49 so that the flow of formulation occupies all or part of the available cross-section of the discharge device 13. Additionally, the shutter device 22 is capable of being partially opened or closed via a solenoid 39 in order to adjust or regulate the flow of formulation. The discharge assembly also includes a nozzle 23 for allowing discharge of the supercritical fluid from device 13. The nozzle 23 can be provided, as necessary, with a nozzle heating module 26 and a nozzle shield gas module 27 to assist in beam collimation. The discharge device 13 also includes a beam control device 24 which encompasses devices such as catchers, stream deflectors, electromagnetic fields, mechanical shields, magnetic lenses, electrostatic lenses, aerodynamic lenses etc. to assist in beam collimation prior to the beam reaching a receiver 14. Components 22-24, 26, and 27 of discharge device 13 are positioned relative to delivery path 16 such that the formulation continues along delivery path 16.

Alternatively, the shutter device 22 can be positioned after the nozzle heating module 26 and the nozzle shield gas module 27 or between the nozzle heating module 26 and the nozzle shield gas module 27. Additionally, the nozzle shield gas module 27 may not be required for certain applications, as is the case with the stream deflector and catcher module 24 and/or the addition of a spacer-shield 25 (see FIG. 1A). To allow the gas portion of the supercritical fluid to escape, a gas release port 38 is attached to the spacer-shield 25 (see FIG. 1A). Alternatively, discharge device 13 can include a stream deflector and catcher module 24 and not include the shutter device 22. In this situation, the stream deflector and catcher module 24 can be moveably positioned along delivery path 16 and used to regulate the flow of formulation such that a continuous flow of formulation exits while still allowing for discontinuous deposition and/or etching.

The nozzle 23 can be capable of translation in x, y, and z directions to permit suitable discontinuous and/or continuous bioactive material deposition and/or etching on the receiver 14. Translation of the nozzle 23 can be achieved through manual, mechanical, pneumatic, electrical, electronic or computerized control mechanisms. Receiver 14 and/or media conveyance mechanism 50 can also be capable of translation in x, y, and z directions to permit suitable bioactive material deposition and/or etching on the receiver 14. Alternatively, both the receiver 14 and the nozzle 23 can be translatabled in x, y, and z directions depending on the particular application.

Referring to FIGS. 2B-2J, the nozzle 23 functions to direct the formulation flow towards the receiver 14. It is also used to attenuate the final velocity with which the bioactive material impinges on the receiver 14. Accordingly, nozzle geometry can vary depending on a particular application. For example, nozzle geometry can be a constant area having a predetermined shape (cylinder 28, square 29, triangular 30, etc.) or variable area converging 31, variable area diverging 38, or variable area converging-diverging 32, with various forms of each available through altering the angles of convergence and/or divergence. Alternatively, a combination of a constant area with a variable area, for example, a converging-diverging nozzle with a tubular extension, etc., can be used. In addition, the nozzle 23 can be coaxial, axisymmetric, asymmetric, or any combination thereof (shown generally at 33). The shape 28, 29, 30, 31, 32, 33 of the nozzle 23 can assist in regulating the flow of the formulation. In a preferred embodiment of the present invention, the nozzle 23 includes a converging section or module 34, a throat section or module 35, and a diverging section or module 36. The throat section or module 35 of the nozzle 23 can have a straight section or module 37.

The discharge device 13 serves to direct the bioactive material onto the receiver 14. The discharge device 13 or a portion of the discharge device 13 can be stationary or can swivel or raster, as needed, to provide high resolution and high precision deposition of the bioactive material onto the receiver 14 or etching of the receiver 14 by the bioactive material. Alternatively, receiver 14 can move in a predetermined way while discharge device 13 remains stationary. The shutter device 22 can also be positioned after the nozzle 23. As such, the shutter device 22 and the nozzle 23 can be separate devices so as to position the shutter 22 before or after the nozzle 23 with independent controls for maximum deposition and/or etching flexibility. Alternatively, the shutter device 22 can be integrally formed within the nozzle 23.

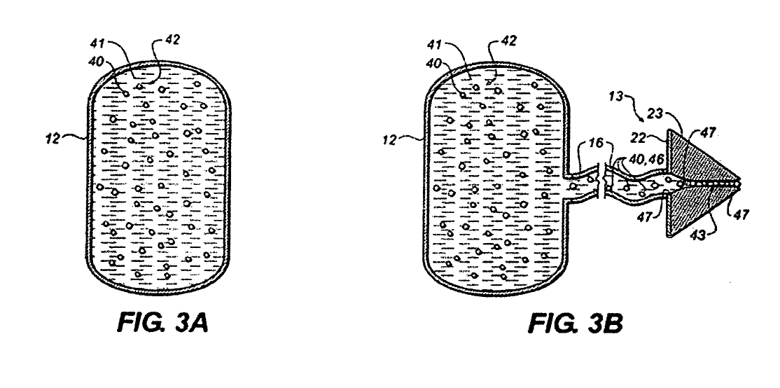

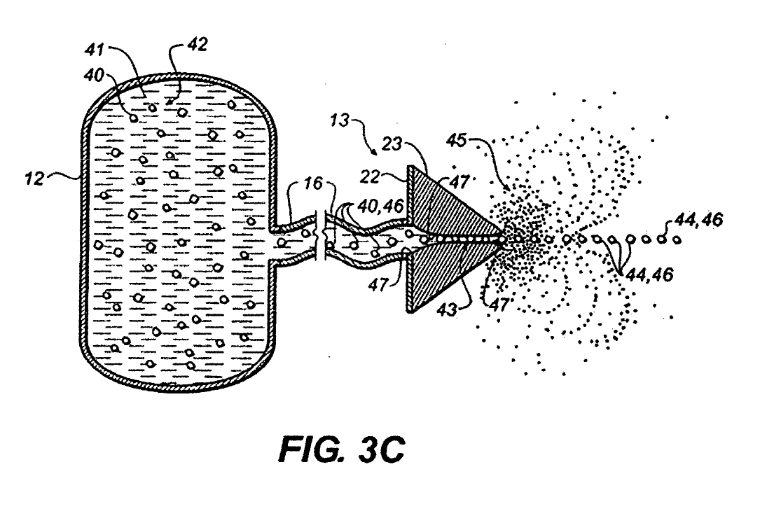

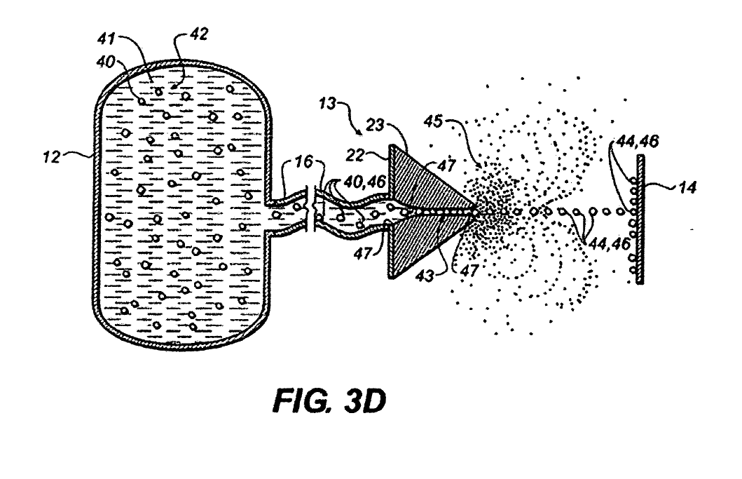

Operation of the delivery system 10 will now be described. FIGS. 3A-3D are diagrams schematically representing the operation of delivery system 10 and should not be considered as limiting the scope of the invention in any manner. A formulation 42 of bioactive material 40 in a supercritical fluid and/or compressed liquid 41 is prepared in the formulation reservoir 12. A bioactive material 40, any material of interest in solid or liquid phase, can be dispersed (as shown in FIG. 3A) and/or dissolved in a supercritical fluid and/or compressed liquid 41 making a mixture or formulation 42. The bioactive material 40 can have various shapes and sizes depending on the type of the bioactive material 40 used in the formulation.

The supercritical fluid and/or compressed liquid 41, forms a continuous phase and bioactive material 40 forms a dispersed and/or dissolved single phase. The formulation 42 (the bioactive material 40 and the supercritical fluid and/or compressed liquid 41) is maintained at a suitable temperature and a suitable pressure for the bioactive material 40 and the supercritical fluid and/or compressed liquid 41 used in a particular application. The shutter 22 is actuated to enable the ejection of a controlled quantity of the formulation 42. The nozzle 23 collimates and/or focuses the formulation 42 into a beam 43 as shown in FIG. 3B.

The bioactive material 40 is controllably introduced into the formulation reservoir 12. The compressed liquid/supercritical fluid 41 is also controllably introduced into the formulation reservoir 12. The contents of the formulation reservoir 12 are suitably mixed using mixing device 70 to ensure intimate contact between the bioactive material 40 and compressed liquid/supercritical fluid 41. As the mixing process proceeds, bioactive material 40 is dissolved or dispersed within the compressed liquid/supercritical fluid 41. The process of dissolution/dispersion, including the amount of bioactive material 40 and the rate at which the mixing proceeds, depends upon the bioactive material 40 itself, the particle size and particle size distribution of the bioactive material 40 (if the bioactive material 40 is a solid), the compressed liquid/supercritical fluid 41 used, the temperature, and the pressure within the formulation reservoir 12. When the mixing process is complete, the mixture or formulation 42 of bioactive material and compressed liquid/supercritical fluid is thermodynamically stable/metastable in that the bioactive material is dissolved or dispersed within the compressed liquid/supercritical fluid in such a fashion as to be indefinitely contained in the same state as long as the temperature and pressure within the formulation chamber are maintained constant. This state is distinguished from other physical mixtures in that there is no settling, precipitation, and/or agglomeration of bioactive material particles within the formulation chamber unless the thermodynamic conditions of temperature and pressure within the reservoir are changed. As such, the bioactive material 40 and compressed liquid/supercritical fluid 41 mixtures or formulations 42 of the present invention are said to be thermodynamically stable/metastable.

The bioactive material 40 can be a solid or a liquid. Additionally, the bioactive material 40 can be an organic molecule, a polymer molecule, a metallo-organic molecule, an inorganic molecule, an organic nanoparticle, a polymer nanoparticle, a metallo-organic nanoparticle, an inorganic nanoparticle, an organic microparticle, a polymer micro-particle, a metallo-organic microparticle, an inorganic microparticle, and/or composites of these materials, etc. After suitable mixing with the compressed liquid/supercritical fluid 41 within the formulation reservoir 12, the bioactive material 40 is uniformly distributed within a thermodynamically stable/metastable mixture, that can be a solution or a dispersion, with the compressed liquid/supercritical fluid 41. This thermodynamically stable/metastable mixture or formulation 42 is controllably released from the formulation reservoir 12 through the discharge device 13 as shown in FIG. 3C.

During the discharge process, still referring to FIG. 3C, the bioactive material 40 is precipitated from the compressed liquid/supercritical fluid 41 as the temperature and/or pressure conditions change. The precipitated bioactive material 44 is directed towards a receiver 14 by the discharge device 13 as a focused and/or collimated beam. The particle size of the bioactive material 40 deposited on the receiver 14 is typically in the range from one nanometer to 1000 nanometers. The particle size distribution may be controlled to be uniform by controlling the rate of change of temperature and/or pressure in the discharge device 13, the location of the receiver 14 relative to the discharge device 13, and the ambient conditions outside of the discharge device 13.

The delivery system 10 is also designed to appropriately change the temperature and pressure of the formulation 42 to permit a controlled precipitation and/or aggregation of the bioactive material 40. As the pressure is typically stepped down in stages, the formulation 42 fluid flow is self-energized. Subsequent changes to the formulation 42 conditions (a change in pressure, a change in temperature, etc.) result in the precipitation and/or aggregation of the bioactive material 40 coupled with an evaporation (shown generally at 45) of the supercritical fluid and/or compressed liquid 41. The resulting precipitated and/or aggregated bioactive material 44 deposits on the receiver 14 in a precise and accurate fashion. Evaporation 45 of the supercritical fluid and/or compressed liquid 41 can occur in a region located outside of the discharge device 13. Alternatively, evaporation 45 of the supercritical fluid and/or compressed liquid 41 can begin within the discharge device 13 and continue in the region located outside the discharge device 13. Alternatively, evaporation 45 can occur within the discharge device 13.

A beam 43 (stream, etc.) of the bioactive material 40 and the supercritical fluid and/or compressed liquid 41 is formed as the formulation 42 moves through the discharge device 13. When the size of the precipitated and/or aggregated bioactive material 44 is substantially equal to an exit diameter of the nozzle 23 of the discharge device 13, the precipitated and/or aggregated bioactive material 44 has been collimated by the nozzle 23. When the size of the precipitated and/or aggregated bioactive material 44 is less than the exit diameter of the nozzle 23 of the discharge device 13, the precipitated and/or aggregated bioactive material 44 has been focused by the nozzle 23.

Referring now to FIG. 3D, the receiver 14 is positioned along the path 16 such that the precipitated and/or aggregated bioactive material 44 is deposited on the receiver 14. As the individual particle size of the precipitated and/or aggregated bioactive material 44 is extremely small, generally less than about 100 nanometers, adhesion forces are sufficient to keep the particles in place on the receiver 14. Preferably the particle size is between 10 and 50 nanometers.

The distance of the receiver 14 from the discharge nozzle 23 is chosen such that the supercritical fluid and/or compressed liquid 41 evaporates from the liquid/carrier and/or supercritical phase to the gas phase (shown generally at 45) prior to reaching the receiver 14. Hence, there is no need for subsequent receiver-drying processes. Further, subsequent to the ejection of the formulation 42 from the nozzle 23 and the precipitation of the bioactive material, additional focusing and/or collimation may be achieved using external devices such as electromagnetic fields, mechanical shields, magnetic lenses, electrostatic lenses etc. Alternatively, the receiver 14 can be electrically or electro statically charged such that the positional placement of the bioactive material 40 on the receiver can be controlled.

It is also desirable to control the velocity and thereby the momentum, with which individual particles 46 of the bioactive material 40 are ejected from the nozzle 23. As there is a sizable pressure drop from within the delivery system 10 to the operating environment, the pressure differential converts the potential energy of the delivery system 10 into kinetic energy that propels the bioactive material particles 46 onto the receiver 14. The velocity of these particles 46 can be controlled by suitable nozzle design and control over the rate of change of operating pressure and temperature within the system. Further, subsequent to the ejection of the formulation 42 from the nozzle 23 and the precipitation of the bioactive material 40, additional velocity regulation of the bioactive material 40 may be achieved using external devices such as electromagnetic fields, mechanical shields, magnetic lenses, electrostatic lenses etc. Nozzle design and location relative to the receiver 14 also determine the pattern of bioactive material 40 deposition. The actual nozzle design will depend upon the particular application addressed.

The nozzle 23 temperature can also be controlled via the nozzle heating module 26 shown in FIG. 2A. Nozzle temperature control may be controlled as required by specific applications to ensure that the nozzle opening 47 maintains the desired fluid flow characteristics. Nozzle temperature can be controlled through the nozzle heating module 26 using a water jacket, electrical heating techniques, etc. With appropriate nozzle design, the exiting stream temperature can be controlled at a desired value by enveloping the exiting stream with a co-current annular stream of a warm or cool, inert gas from the nozzle shield module 27, as shown in FIG. 2A.

The deposition characteristics of the bioactive material 40 are a function of several factors including the bulk modulus of the receiver 14, the bulk modulus of the bioactive material 40, density of the receiver 14, the density of the bioactive material 40, the pressure-difference between the formulation reservoir and ambient conditions, the temperature difference between the formulation reservoir and ambient conditions, the deposition time, the discharge nozzle geometry, the distance between the discharge nozzle and the receiver, bioactive material size and momentum, etc. These factors can be modified or held constant depending on the application. For example, in a printing application wherein the bioactive material 40 is to be deposited on the receiver surface, the nozzle geometry, formulation conditions, ambient conditions, and bioactive material can be fixed. The deposition of the bioactive material 40 can then be controlled by altering the receiver design (e.g. the bulk modulus of the receiver, the distance between the discharge nozzle and the receiver, the deposition time, etc.). Alternatively, for the same application, it is possible to alter formulation conditions (e.g. bioactive material concentration, etc.). Alternatively, for a printing application wherein the bioactive material 40 is to be deposited within the receiver, the deposition can be controlled by altering the receiver design (e.g. the bulk modulus of the receiver, formulation conditions, etc.), while keeping the other parameters fixed.

For a given constant nozzle geometry, constant conditions within the formulation reservoir, unchanging ambient conditions, constant deposition time, and a constant distance between the tip of the discharge nozzle and the receiver, the main receiver property that governs the accuracy of deposition of the bioactive material 40 is the receiver bulk modulus relative to the bioactive material bulk modulus. The bulk modulus of a material, typically expressed in Pascals, is a measure of its compressibility or its ability to absorb the momentum of a particle. Specifically, it is a measure of the change in volume of the material as the pressure is changed. It may be expressed isothermally or adiabatically. The isothermal bulk modulus is specified in this application.

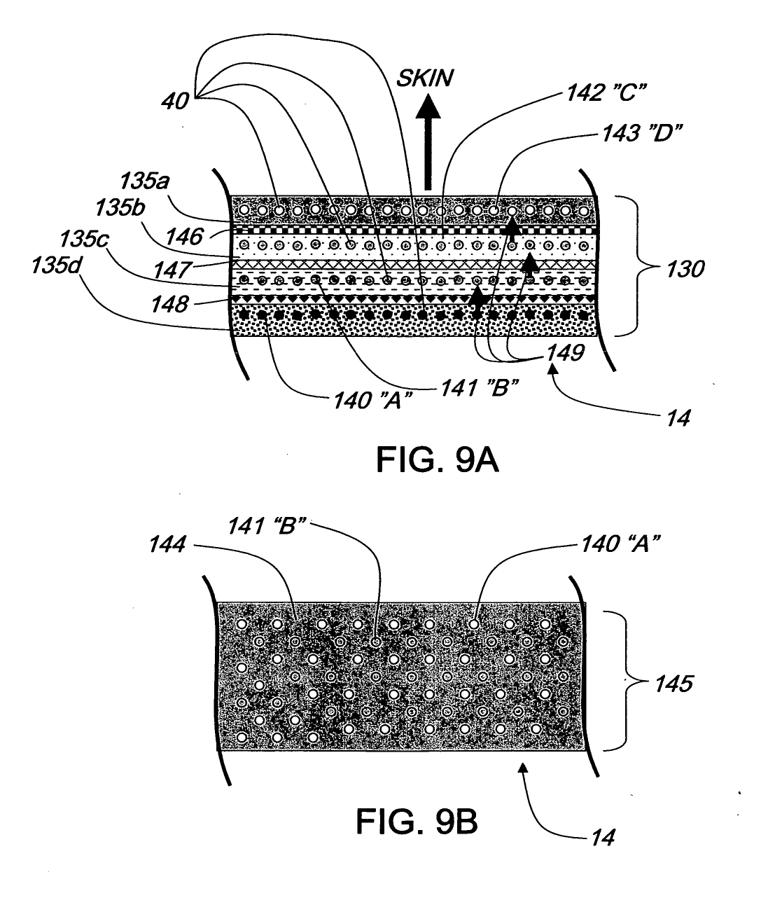

The receiver can be a single layer as described in FIG. 9B or multi-layer receiver as described in FIG. 9A having one or more layers with a bulk modulus of between 10 Mpa and 100 GPa positioned at a distance between 0.01 cm and 25 cm from the nozzle of the discharge device. For example, by using cross-linked gelatin as a receiver or donor, its modulus will depend on the relative humidity and its contact with water. For instance, one can control the penetration of the particles with the force of the CO2 application or keep the force constant and change the modulus of the gelatin with relative humidity (RH) and thus change the penetration depth as shown in the following graph.

[image]

Pascals can be converted to psi by multiplying by 1.45×10-4. One could also change the modulus by changing the gel layer structures as later described in FIG. 9A. The choice of receiver bulk modulus also depends on the bioactive material bulk modulus. With all other parameters held constant, if the receiver bulk modulus is significantly larger than that of the bioactive material, it can be reasonably expected that the bioactive material particles are significantly altered in shape upon impact with the receiver 14. Alternatively, when the bioactive material bulk modulus is much higher than that of the receiver, the bioactive material particles may retain much of their original shape even after impact with the receiver 14.

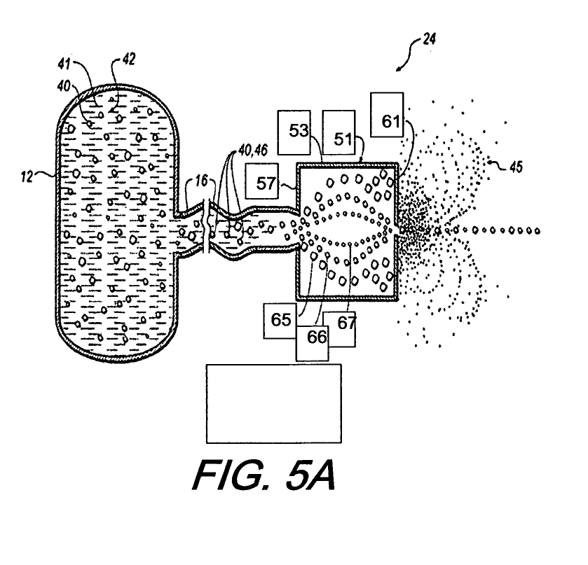

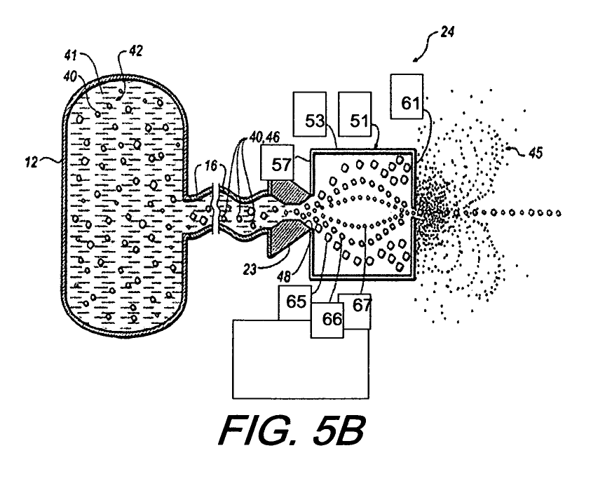

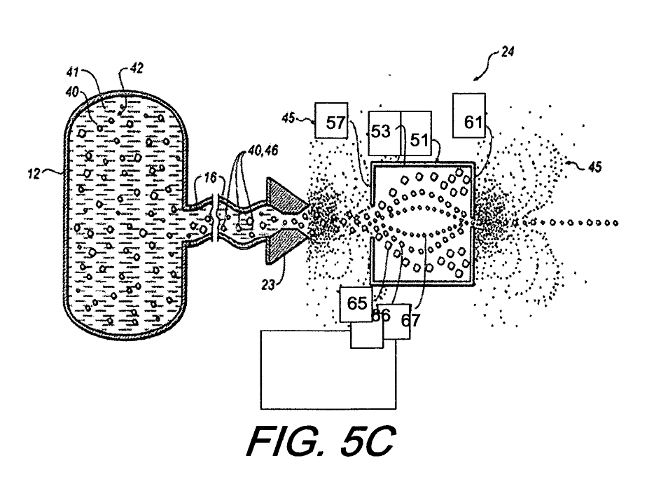

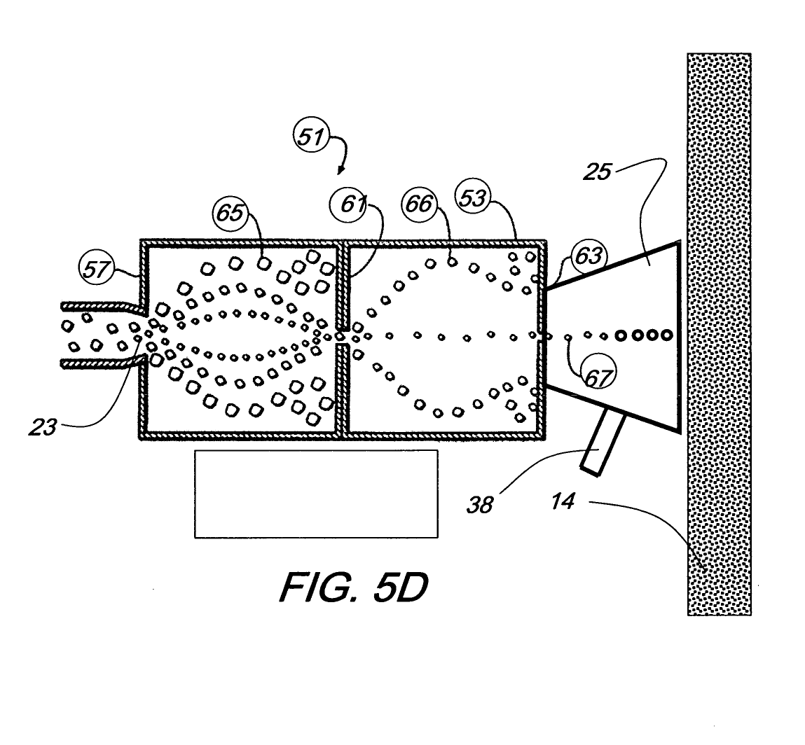

Referring to FIGS. 5A-5C, subsequent to the ejection of the formulation 42 from the nozzle 23 and the precipitation of the functional bioactive material 40, additional velocity regulation, focusing, and/or directioning of the functional material 40 can be achieved using the beam control device 24 and spacer-shield 25. The spacer-shield 25 locates the nozzle 23 the appropriate distance from the receiver 14 and acts as a catch module. As previously described, the spacer-shield 25 may be attached to the gas release port 38 or be made of a porous material to allow the gas portion of the supercritical fluid to be dissipated. The beam control device 24 includes devices such as catchers, stream deflectors, electromagnetic fields, mechanical shields, magnetic lenses, electrostatic lenses, aerodynamic lenses etc. The location of beam control device 24 can vary. The beam control device 24 can be part of the discharge device 13, either integrally formed or attached thereto and may, in certain instances, take the place of the spacer-shield 25. Alternatively, the beam control device 24 can be spaced apart from the discharge device 13.

When the beam control device 24 is an integral part of the discharge device 13, the functional material 40 is formed as the formulation moves through the beam control device 24. In this respect, the beam control device 24 can function as a focusing nozzle. As such, the nozzle 23 of the discharge device 13 can be replaced by the beam control device 24, as shown in FIG. 5A.

When additional focusing of the functional material is desired, the beam control device 24 can be positioned at the outlet 48 of the nozzle 23, as shown in FIG. 5B. When the beam control device 24 is positioned in this manner, the functional bioactive material 40 is formed as the formulation moves through the beam control device 24.

Alternatively, the beam control device 24 can be spaced apart from the nozzle 23 positioned in the material delivery path 16, as shown in FIG. 5C. When the beam control device 24 is positioned in this manner, the beam of functional bioactive material 40 is formed and then focused by passing it through the beam control device 24.

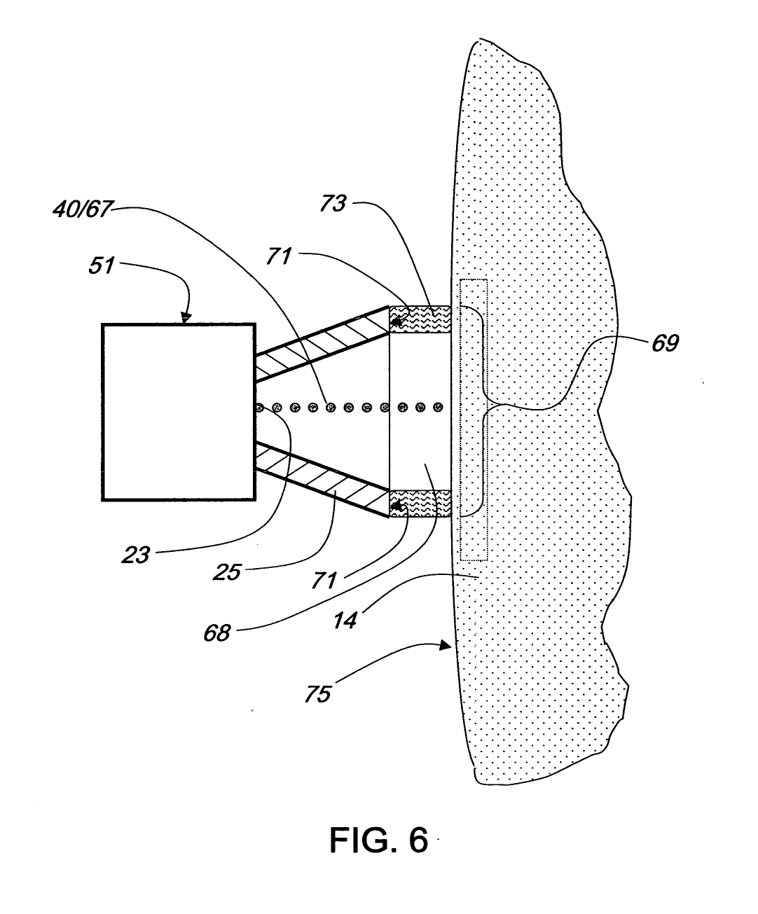

In one embodiment, a spacer-shield shown in FIGS. 5D and 6 is also provided between the discharge nozzle 23 and a cutaneous target, to space the delivery system a desired distance away from the cutaneous target during delivery of the bioactive material. This spacer-shield may be attached to either the skin or the delivery system, or merely be interposed between them, to provide an interface across which the bioactive material may be distributed from the orifice, or from an array of orifices, to a target. The target may include skin or a skin patch, such as a transdermal drug delivery patch, which acts as a reservoir for subsequent prolonged transdermal delivery of the material. Additionally, the delivery system to receiving surface spacing advantageously protects the delivery system from unnecessarily coming into contact with the receiver, which avoids debris from the surface of the patch into the dispenser nozzle. Adequate spacing between the nozzles and patch also avoids inadvertent or unwanted administration of drug to the patch. Such debris or other fibers in the nozzles could potentially damage the nozzles, leading to fully or partially blocked nozzles that dispense less bioactive material than intended. Such debris could also lead to misdirected droplets that would miss the target area.

Again referring to FIGS. 5A-5C and referring to FIG. 5D, the beam control device 24 can be, for example, an aerodynamic lens 51. Aerodynamic lens 50 includes a tubular pipe (capillary, etc.) 53 having one or more orifice plates 57, 61, 63 with diameters smaller than the tubular pipe 53 positioned along the delivery path 16 such that additional focusing of the beam of functional bioactive material 40 occurs. Additional focusing occurs as the functional bioactive material 40 passes through the aerodynamic lens 51 because the orifice plates 57, 61, 63 are sized to prevent particles 65, 66 of functional bioactive material 40 from passing through the aerodynamic lens 51 (as shown in FIG. 5D) while particles 67 are permitted to pass through aerodynamic lens 51. In FIGS. 5A-5D, particles 65 and 66 are larger in size when compared to particles 67. The specific diameters of the orifice plates 57, 61, 63 will depend on the desired particle size of the functional material. Additional orifice plates can also be added depending on the desired particle size.

Alternatively, the aerodynamic lens 51 can include a first capillary tube of a given diameter in fluid communication with a second capillary tube of smaller diameter. These capillary tubes can also include one or more orifice plates 57, 61, 63 with smaller diameters.

The interface provided by a spacer-shield 25 between the orifice plate 57, 61, 63 and the target allows chemical reactions to occur, as well as phase changes to stabilize (such as a change from a solid to a liquid state). This interface may also provide flexibility in the distribution of the bioactive material 40, pharmaceutical composition, or drug across a larger target area, as compared to application of the bioactive material 40 from an orifice that abuts the target. Using existing solvent-less technology, distribution of the bioactive material 40 to the target may be carefully controlled, and exact dosing of the bioactive material 40 may be achieved. Controllers may be used to dispense simple or complex drug regimens, which is of particular advantage in patients 61 who require numerous daily medications. Computerized control of medication dosing, which may be programmed by medical personnel for subsequent automated delivery, can help avoid toxic drug interactions, overdosages, and deaths. Computerized control can mitigate against the forgetfulness of patients 61.

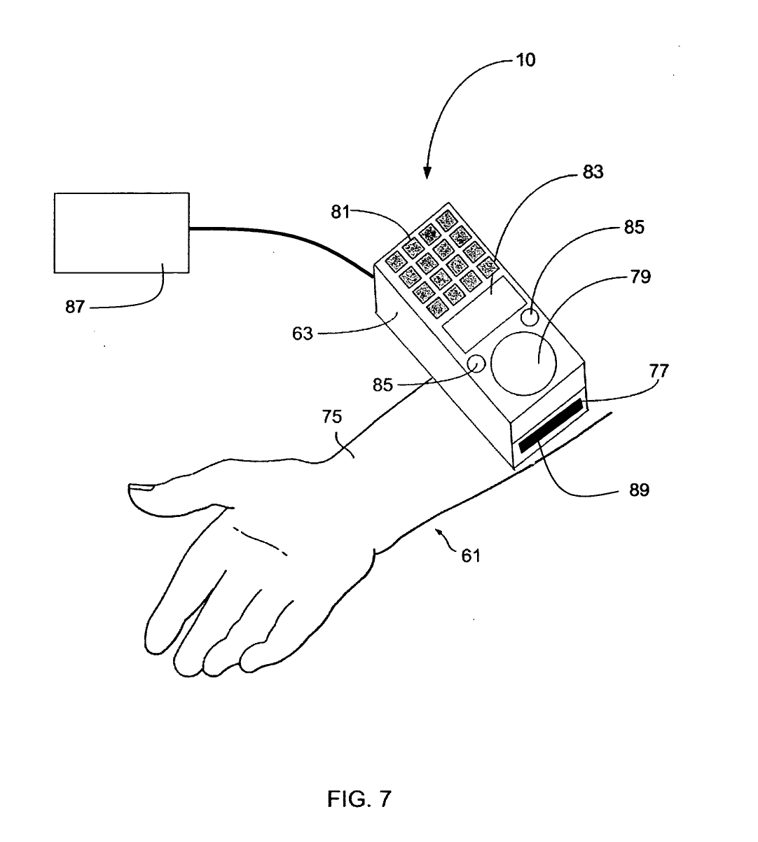

Referring to FIGS. 6 and 7, in some embodiments the delivery system 10 forms a substantially sealed chamber 68 directly against the skin 75, without an intervening transdermal patch, and effectively become a direct cutaneous or transdermal applicator 63 as described in FIG. 7. In particularly effective embodiments, an elastomeric seal 69 is provided between the delivery system 10 and the skin to form the sealed chamber 68 in which the bioactive material 40 can be maintained until it is absorbed, or directed at high velocities, through the stratum corneum layer of the skin. Conditions in the sealed chamber 68 may be altered to enhance absorption, or penetration of the bioactive material 40 or drug, for example by increasing humidity in the chamber by dispensing water droplets, or intermittently applying a penetration enhancer to the skin from the delivery system, or to prepare the skin for penetration by dispensing an anesthetic, anti-inflammatory, or antibiotic bioactive material.

The spacer-shield 25 may be carried by the delivery system 10 and positioned to be disposed against the cutaneous target (skin) while the delivery system 10 ejects the bioactive material 40 such as the pharmaceutical composition from the delivery system 10. A programmable microprocessor 77 in the transdermal applicator 63 may control ejection of the pharmaceutical composition from the delivery system 10 at pre-selected time intervals, such as every three or four hours, or even every few minutes or seconds, or ejection can be triggered by a sensor or other feedback mechanism.

FIG. 6 illustrates an embodiment of the spacer-shield 25 made in accordance with the present invention, which is included to prevent the loss of the bioactive material, pharmaceutical composition, or drug to the surrounding environment. In a preferred embodiment, this shield 21 is wrapped on the inside edge 71 with a flexible porous plastic material 73 which after delivery provides a convenient method of containing and presenting all of the bioactive material 67 to the receiver 14 for example a predetermined layer of the skin 75. As described by Bellhouse et al., and Bell et al., in U.S. Pat. No. 6,685,669 B2, direct precutaneous drug delivery from the above-described system is generally practiced using particles having an approximate size generally ranging from 0.1 to 250 μm. For drug delivery, the optimal particle size is usually at least about 10 to 15 μm (the size of a typical cell).

Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematics viscosity of the targeted skin or mucosal tissue. In this regard, optimal particle densities for use in needleless injection generally range between about 0.1 and 25 g/cm³, preferably between about 0.9 and 1.5 g/cm³, and injection velocities can range from about 200 to about 3,000 m/sec.

The above mentioned conditions describe the physical properties necessary to allow bioactive materials to overcome the barrier properties, or the modulus of elasticity, of the stratum corneum layer of human skin. There is a great deal of variability in the modulus of elasticity of human skin. Factors influencing this would include age, sex, and location on the body. McElhaney et al. in the book “Handbook of Human Tolerance”, lists the effects of age on the modulus of elasticity

| Age | Modulus of Elasticity (kg/mm\hat{ }2) |
|---|---|
| 7 months-3 years | 2.9 |
| 15 years-30 years | 6.7 |
| 30 years-50 years | 8.1 |
| 50 years-80 years | 11.0 |

* Note -
the above information (modulus of elasticity data) has not been verified through the original text, library search is in progress.

As shown by FIG. 7, the delivery system 10 is suitable for use in a variety of ways. For example, the applicator 63 may be intermittently applied to the skin 75 to administer a dosage of a drug directly to the skin 75 as described above. Alternatively, the applicator 63 may be applied to the transdermal patch 92 to recharge it with medication, instead of replacing the patch. In another embodiment, the applicator 63 may be selectively retained in prolonged contact with the cutaneous target, for example by securing the applicator 63 to the skin 75 with an attachment member, such as a strap (not shown) or adhesive. In this manner, the bioactive material 40 may be administered from the delivery system 10 for a prolonged period of time into a transdermal patch 92, or directly onto the skin 75. A replaceable container module 79 may be removed from the applicator 63 and replaced, to avoid the necessity of removing the applicator 63 from the patient 61.

The delivery system 10 and applicator 63 may include the programming module 77, such as a keypad 81 for entering dosage information, a display screen 83 for showing what information has been entered on a display 100, and indicators 85 (such as one or more lights or a display screen on the exterior of the device) that provide information about how much drug remains in the device. Display screens 100 may also provide information about medications in the device, and provide an interface through which other information about the medications or their administration can be entered and/or obtained. The display may also provide information obtained by various sensors and/or monitors that may be provided in system 10. The sensors and/or monitors are used to monitor the subject to which the bio-active material is being applied. For example, but not limited to, the sensors may comprise a pulse oximetry sensor 91 for monitor pulse rate of the subject and oxygen levels in the blood of the subject. It is of course understood that various other sensors/monitors may be used to monitor other subject parameters. The information obtained by the sensors may be used as feed back for microprocessor 90 for controlling dispensing of the bioactive material by applicator 63. This may be in the form of a signal that generates a response in the delivery of the bio-active material. The sensors 91 may be optical sensors and may communicate the obtained information to the processor 90 by direct connection or by wireless infrared or radio communication. The system 10 may also be provided with audio and/or visual alarms to advising the status of the subject or of the operational conditions of the system 10.

The delivery system 10 may also include memory associated in the processor of a separate memory device 87. The memory may of course be used for storage of information or control data entered by the user or for storing of computer applications for controlling delivery system and its associated components. In addition, a removable memory 89, such as a memory card, may be received by the appropriate slot, which the computer/microprocessor can access for obtaining stored information for computer programs thereon. In this way the delivery system 10 may obtain new information for operating a new bioactive material and/or provide updated information for operating the delivery system 10 with respect to current or new bioactive materials.

The device 63 may be provided as part of a kit to be used by a subject. In addition to the device 63, instructions as to use may also be provided. Also transdermal patches may also be provided on which various bioactive material may be applied by device 63. The transdermal patch would then be applied to the subject. The kit may also include various other sensors and monitors to assist the user of the kit.

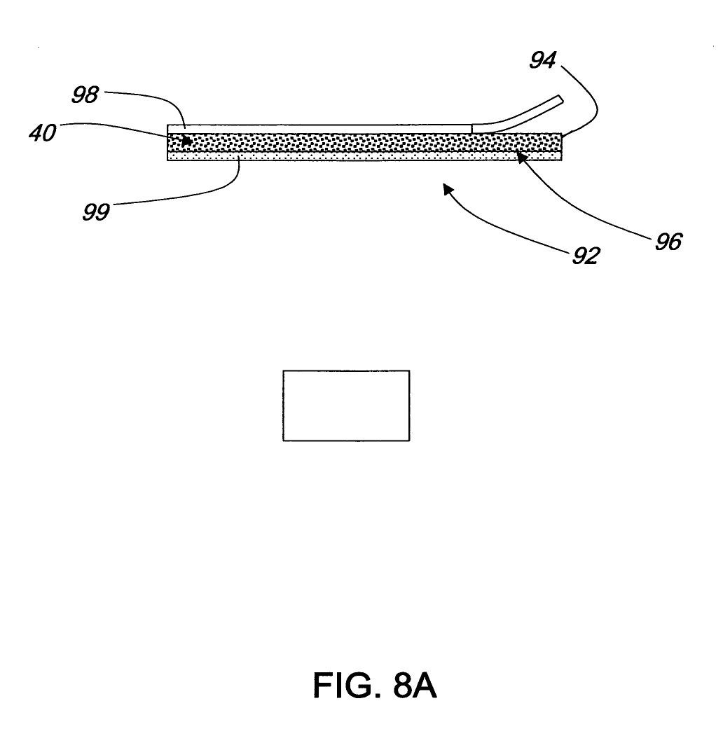

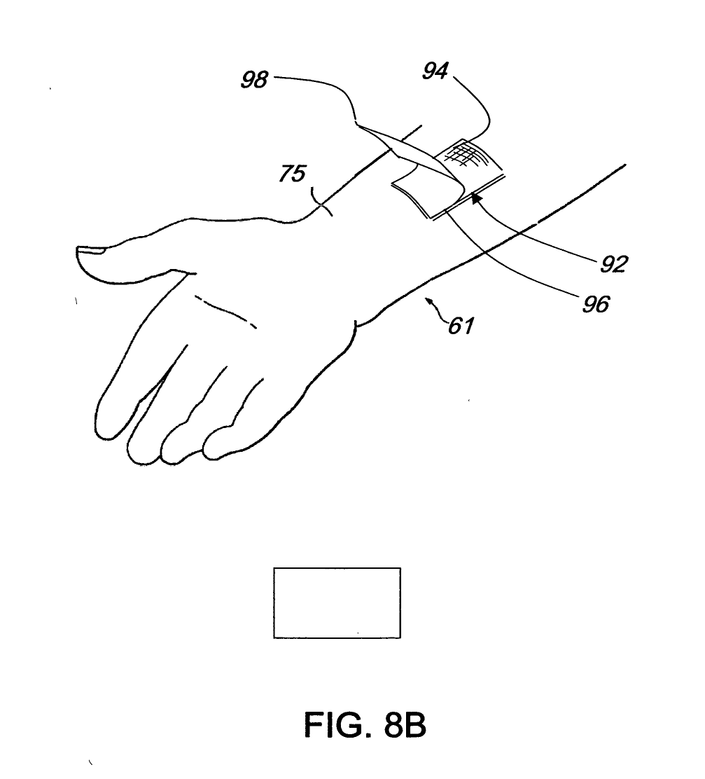

While the bioactive material 40 may be applied directly to skin surface 75 as shown in FIGS. 6 and 7, the receiver 14 illustrated in FIG. 8A, is a transdermal patch 92. By applying the bioactive material 40 to an absorbent member, such as the transdermal patch 92 of a fabric or other absorbent material which may be adhered to skin surface 75 as shown in FIG. 8B, the bioactive material is applied in an indirect manner to the skin. Patch 92 has an upper exposed surface 94, and an opposing under surface 96 which is in contact with skin 75. A removable protective layer 98, such as a layer of a liquid impermeable thin polyester, may be selectively removed and reapplied to patch 92. In one particular embodiment, the fluid is applied to patch 92, which then allows skin 75 to gradually absorb the fluid from patch 92.

Any of the many types of transdermal patches may be used, or modified for use with the delivery system. For example the Testoderm® transdermal system (Alza Pharmaceuticals) uses a flexible backing of transparent polyester, and a testosterone containing film of ethylene-vinyl acetate copolymer membrane that contacts the skin surface and controls the rate of release of active agent from the system. The surface of the drug containing film is partially covered by thin adhesive stripes of polyisobutylene and colloidal silicon dioxide, to retain the drug film in prolonged contact with the skin. In the present system, adhesive can be provided on both surfaces of the drug containing film, for example on both upper face 94 and under face 96 of patch 92, so that the flexible polyester backing 98 may be selectively removed to provide access to the drug-containing layer without removing the patch.

An adhesive release layer with openings in it can be provided between the patch 92 and backing 98, to help protect upper face 94 of patch 92 during repeated removals of backing 98. Alternatively, the patch 92 may be removed, recharged with the drug, and then reapplied, in which event the impermeable backing 98 may be permanently applied to patch 92. In this case, an adhesive layer 99 need only be present under surface 96 of patch 92. In yet other embodiments, there may be no impermeable backing, such as layer 98, over patch 92, so, for instance the selected drug may be continually administered, or the absorbency of the patch is sufficient to retain the drug in the patch without an impermeable backing. Further examples of transdermal patches that may be used or modified for use in the present system and method include the Nicoderm®, Latitude™ and Duragesic® patch.

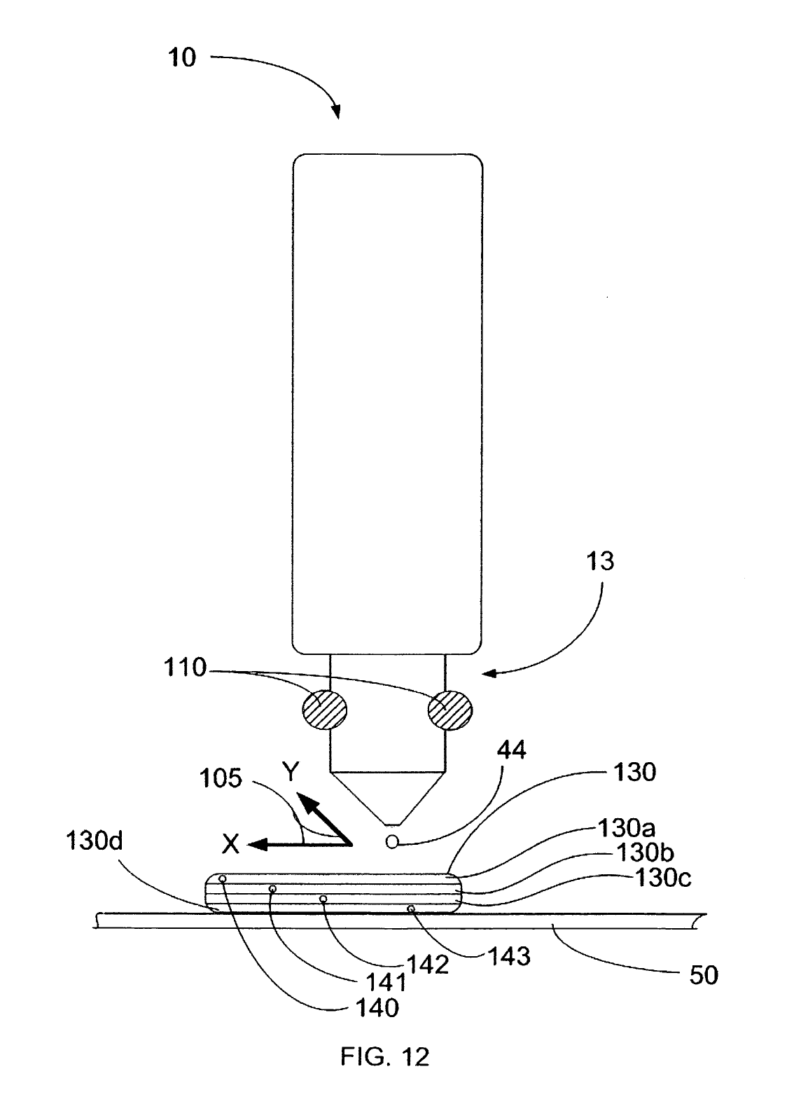

FIG. 9A illustrates another embodiment of the receiver 14, where receiver 14 is a multi-layer time-release material 130. In the example illustrated in FIGS. 9A and 12 there are four separate time release layers 135a, 135b, 135c, and 135d. To achieve time release, the delivery system 10 delivers the bioactive material 40 as previously described so that each individual bioactive material particle 140, 141, 142, and 143 is deposited into its appropriate time release layer 135a, 135b, 135c, and 135d. The receiver 14 can comprise multiple layers of varying bulk moduli as previously discussed. In applications in which the bioactive material 40 is to be located in a layer other than in the top layer, receiver layers of varying bulk moduli may be selected and layered in such a fashion as to allow the bioactive material 40 to penetrate through the top layer or layers and into the layer of choice. The multi-layer time-release material 130 is comprised of several individual layers. The diffusivity of the drugs into and through each of the layers can be controlled by the type of material in each layer. Where one material such as cross-linked gelatin is used for all the time release layers 135a, b, c, and d, semipermeable layers 146, 147 and 148 may be placed between each of the time release layers to control the diffusion rate of the time release bioactive particles “A” 140, “B” 141, “C” 142 and “D” 143 through the layers 135a, b, c, and d respectively. Semipermeable layer 146 is permeable to bioactive particles “B” 141, “C” 142 and “D” 143 but not permeable to bioactive particle “A” 140. Likewise semipermeable layer 147 is permeable to bioactive particles “C” 142 and “D” 143 but not permeable to bioactive particle “B” 141, and semipermeable layer 147 is permeable to bioactive “D” 143 but not permeable to bioactive particle “C” 142. Using the semipermeable layers, the timed diffusion of the time release bioactive particles “A” 140, “B” 141, “C” 142 and “D” 143 to the skin can be controlled as indicated by the arrows 149.

Similarly, in the case of an orally taken time release medicine, semipermeable layers can also be used to control the release of bioactive particles into the stomach and intestines, however the diffusivity can be controlled via the RH at which time the release layer was formed as described above. Suppose the diffusivity at a given RH of each of the drugs composed the bioactive particles in both of the layers was the same. The patient would start seeing bioactive particle “A” 140 first and then bioactive particle “B” 141 once the diffusion was triggered by RH or contact.

FIG. 9B illustrates another embodiment of the receiver 14, where transdermal delivery system is a single layer time-release material 130. The receiver 14 supports a single layer of adhesive 144 serving also as a carrier for the bioactive material 40. In applications in which the bioactive material 40 is to be distributed to the host in a time release fashion, the adhesive-time release carrier material 144 controls, or assists in the control of the migration of the bioactive material 40 through the single layer into the host. In another embodiment both bioactive particle “A” 140 and bioactive particle “B” 141 could also be put in one layer, or bioactive particle “A” 140 could be put in layer two (not shown) and mitigate its delivery by controlling its diffusion through layer one. Any one of these methods could be used to control the diffusion of the bioactive particle or drug to achieve the appropriate time release.

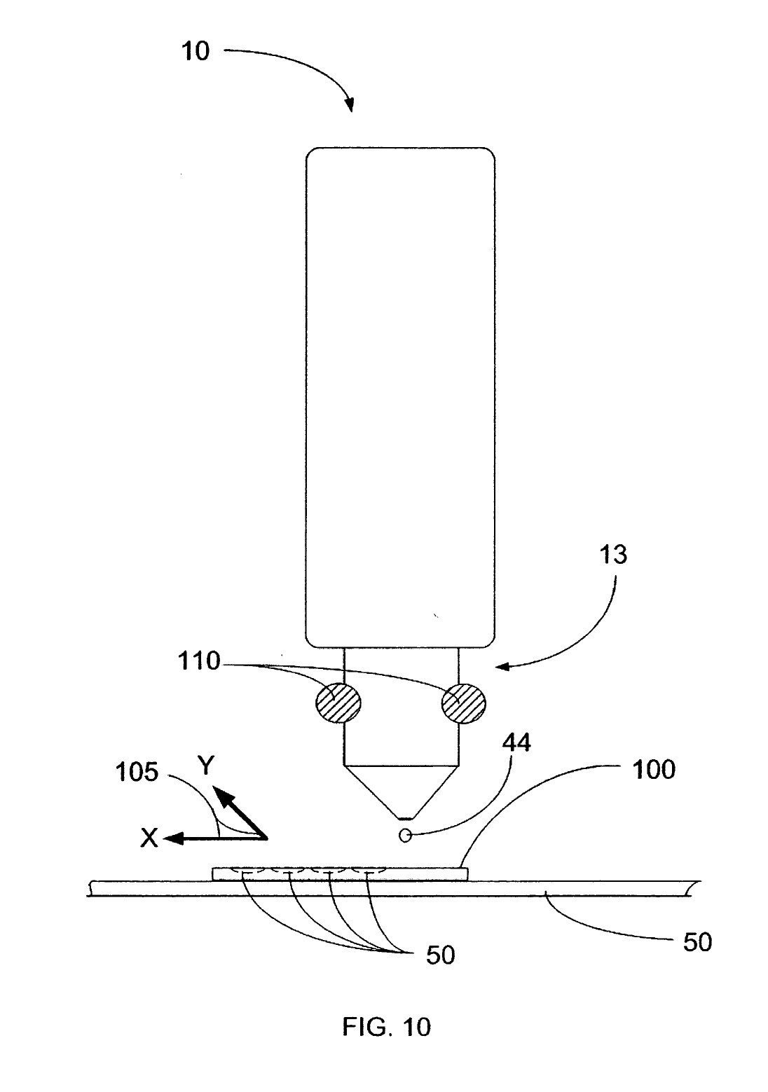

FIG. 10 illustrates another embodiment of the delivery system 10 that delivers the bioactive material 40 onto the receiver 14 where the receiver 14 is a transdermal patch 100. The discharged device 13 is moved in the “x” direction indicated by arrow 105 by the conveyance mechanism 50 and in the “y” direction by a translation device 110.

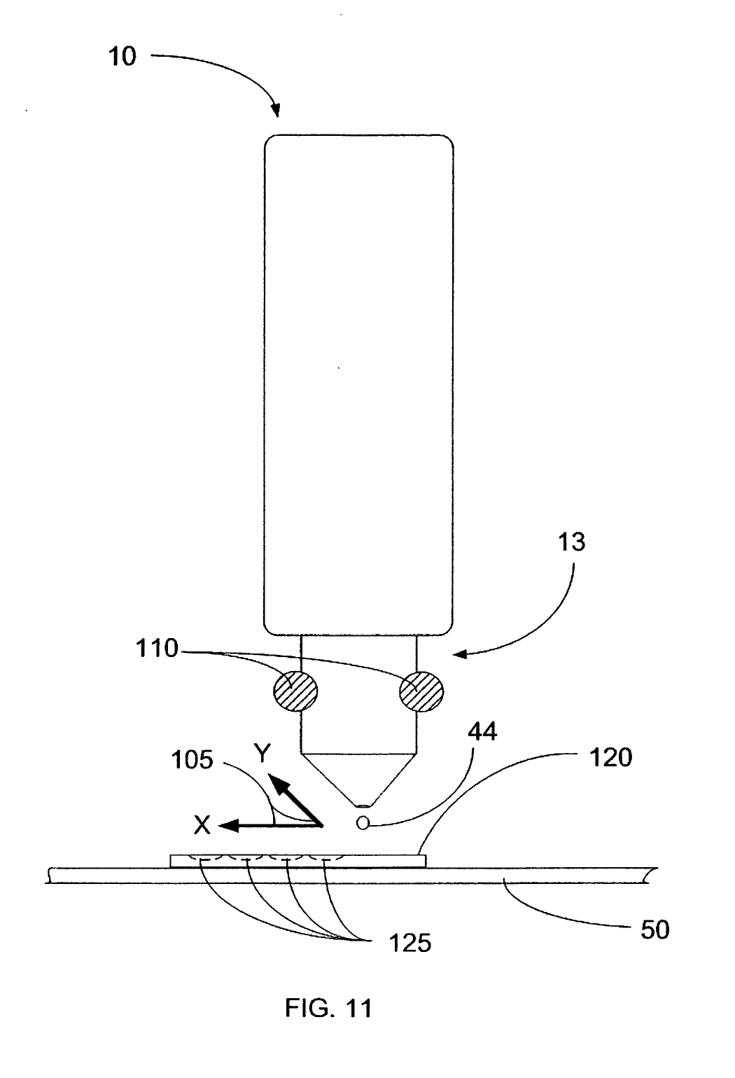

FIG. 11 illustrates another embodiment of the delivery system 10 that delivers the bioactive material 40 onto the receiver 14 where the receiver 14 is freeze-dried gelatinous oral material 120. The discharged device 13 is moved in the “x” direction indicated by arrow 100 by the conveyance mechanism 50 and in the “y” direction by a translation device 110. The bioactive material particles 125 are embedded into the freeze-dried gelatinous oral material 120. The pharmaceutical is one then that may be taken orally.

FIG. 12 illustrates yet another embodiment of the delivery system 10 that delivers the bioactive material 40 onto the receiver 14 where the receiver 14 is a multi-layer time-release material 130 as previously described in FIG. 9A. The multi-layer time-release material 130 is comprised of several individual layers. In the example illustrated in FIG. 12 there are four separate time release layers 135a, 135b, 135c, and 135d. To achieve time release, the delivery system 10 delivers the bioactive material 40 as previously described so that each individual bioactive material particle 140, 141, 142, and 143 is deposited into its appropriate time release layer 135a, 135b, 135c, or 135d. The discharged device 13 is moved in the “x” direction indicated by arrow 100 by the conveyance mechanism 50 and in the “y” direction by a translation device 110. The pharmaceutical laden receiver 14 is then applied to the skin for transdermal dosing. In a second embodiment the receiver 14 is taken orally, and in a third embodiment the receiver 14 is taken as a suppository.

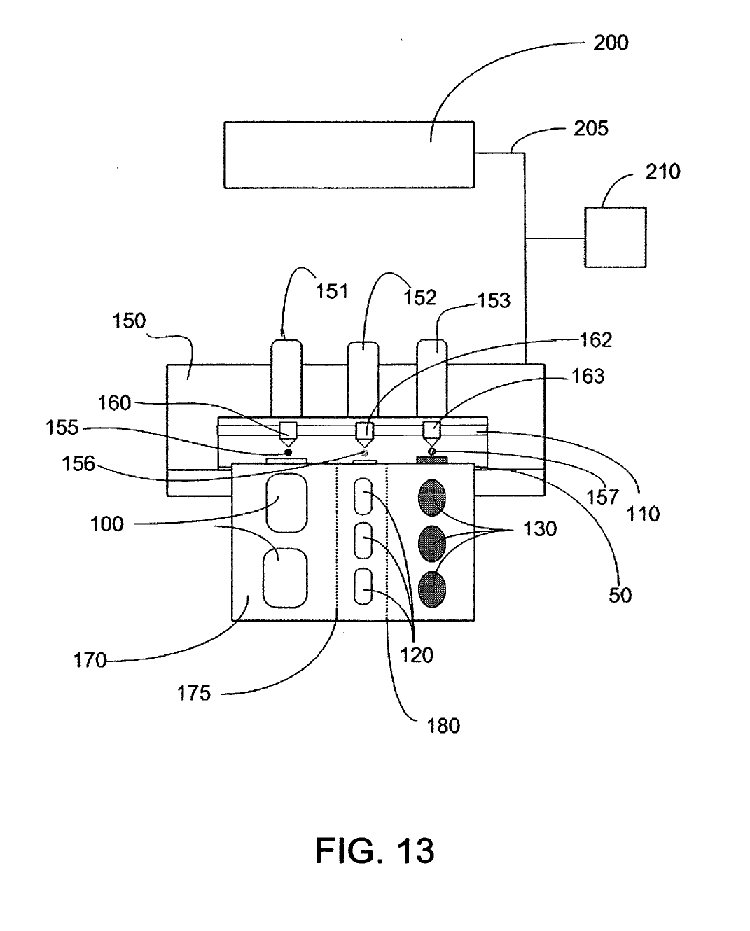

FIG. 13 illustrates a multiple delivery system 150 comprising three separate delivery systems 151, 152, and 153 capable of depositing the appropriate bioactive material 155, 156, and 157 onto transdermal patch 100, freeze-dried gelatinous oral material 120, and multi-layer time-release material 130 respectively. Each of the separate delivery systems 10 of the multiple delivery systems 151, 152, and 153 has its own discharge device 160, 161, and 162, and translation device 110, but may share one conveyance mechanism 50, which conveys a web 170 containing the three types of receivers. The web 170 may be separated into three separate webs along perforations 175 and 180. A logic and control unit 200 connected via cable 205 may control the multiple delivery system 150. The multiple delivery system 150 may also be connected to a communications network such as the Internet via a modem 210. It is apparent that other communication devices may be used to communicate between external computing with the controller 200, such as by using infrared signals, radio waves, and the like.

Alternatively, the controller 200 can be used to adjust dosages of drug administered, for example for a particular time of day, an event (such as an activity that will require a dosage modification), or detection of a physiological condition (such as an adverse drug reaction that requires reduction or cessation of drug administration). When the delivery system 10 is used with a patch 100, the delivery system may be used to recharge the patch and avoid the necessity of changing the patch as often. Either with or without a patch, complex administration protocols may be followed, for example applying different drugs at different times throughout the day or longer period, for example as long as a week, a month, or even longer.

A number of use modes can be envisioned, for example, a patient can download information stored in the device about self-regulated dosage administrations or symptoms experienced (as indicated for example by which buttons have been depressed on the device, and/or the pattern and frequency of the buttons that are pushed). This information can be transmitted over a modem to a physician's or other health care provider's office, where it can be displayed (in electronic or other form) to a health care professional, and appropriate action can be taken. For example, if symptoms are noted to be increasing in spite of administration of a therapeutic amount of a particular drug, consideration can be given to providing a new drug or reconsidering the diagnosis for which the drug has been administered.

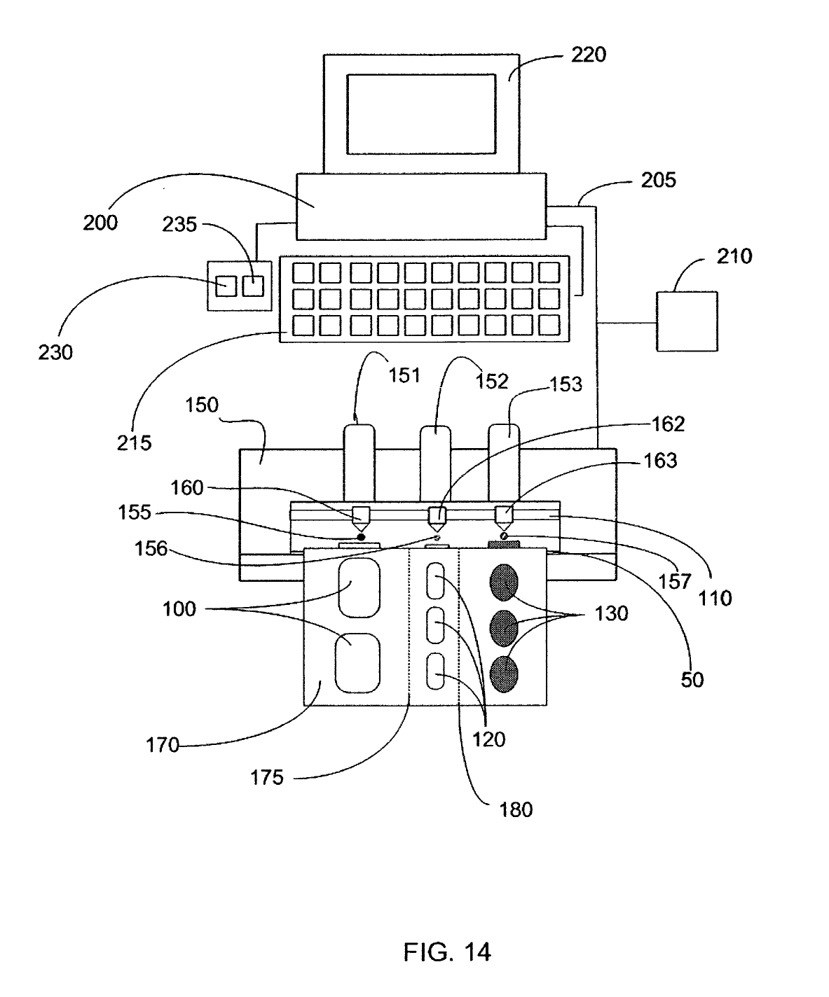

As illustrated in FIG. 14, the multiple delivery system 150 includes a delivery system controller 200, illustrated schematically for convenience. Controller 200 and delivery systems 151-153 receive power from an onboard battery storage system not shown.

Alternatively, power may be supplied from an external source, such as a standard electrical outlet not shown. Of course, rechargeable or replaceable batteries may be preferred in some embodiments for ease of portability and use. Controller 200 operates to apply firing signals to the delivery systems 151, 152, and 153, which respond by delivering bioactive materials.

In a more sophisticated embodiment shown in FIG. 12, controller 200 may include an input keyboard 215, such as an alpha or alpha numeric keypad. Using keyboard 215, a physician, nurse, pharmacist or other health professional, or the subject may input variations in the amount of and types of fluids dispensed. Controller 200 may also include a display screen 220, such as liquid crystal display, to indicate which selections have been made using keyboard 215.

Alternatively, keypad 215 may be eliminated and the controller 200 programmed to display various selections on screen 220. Use of a pair of scrolling buttons 230 and 235 may allow different instructions or selections to be scrolled across, or up and down along, screen 220, including such information such as desired dosages, frequency, and potential side effects.

Display screen 220 may also indicate various selections along an upper portion of the screen allowing a user to then select a particular drug, dosage or delivery method. Alternatively, selecting a particular drug or dosage could indicate the occurrence of a particular event, such as an adverse medication response that would alter (for example decrease) a subsequent dosage administration, or an event (such as physical exertion) that can signal a need to alter a medication dosage. The controller can also be programmed to prevent unauthorized alteration of dosages, for example an increase in a dosage of a controlled substance above that authorized by the prescribing physician. Alternatively, the controller can permit certain ranges of dosages to be administered, for example various doses of an opioid pain reliever in response to fluctuating pain.

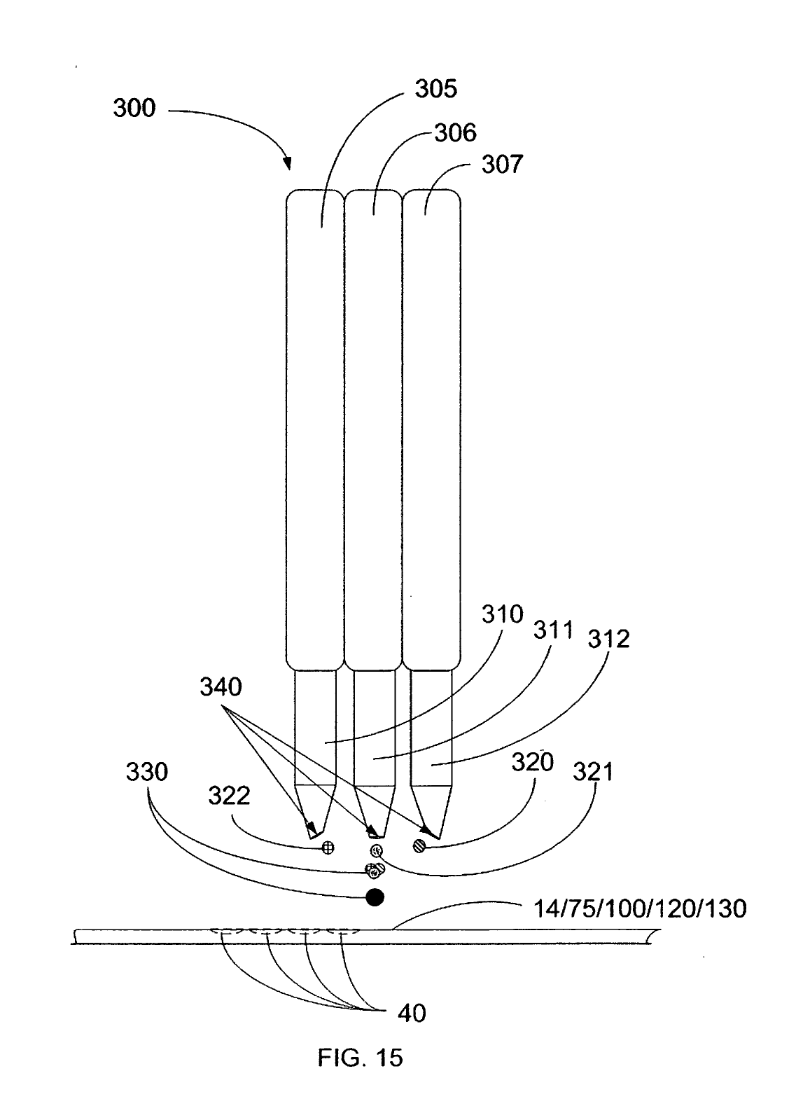

In certain examples as illustrated in FIG. 15, the delivery system 300 may carry multiple container modules 305, 306, and 307 such as removable and replaceable modules each operatively connected to its own discharge device 310, 311, 312 respectively that contain the bioactive material(s) 40. Several modules may contain the same or different materials, for example different materials 320, 321, 322 that combine 330 after being ejected from the discharge devices 310, 311, 312 but before or at the time of delivery to modify one or more of the materials, or to produce a desired bioactive effect, when delivered to the receiver 14 where the receiver 14 may be the skin 75, transdermal patch 100, freeze-dried gelatinous oral material 120, or multi-layer time-release material 130. An example of a modifying substance that may be combined at the point of discharge 340 or the discharge devices is a penetration enhancer that improves cutaneous penetration of the other bioactive material. Penetration enhancers that may be mixed with a bioactive material at the time of delivery include solvents such as water; alcohols (such as methanol, ethanol and 2-propanol); alkyl methyl sulfoxides (such as dimethyl sulfoxide, decylmethyl sulfoxide and tetradecylmethyl sulfoxide); pyrrolidones (such as 2-pyrrolidone, N-methyl-2-pyrroloidone and N-(2-hydroxyethyl)pyrrolidone); laurocapram; and miscellaneous solvents such as acetone, dimethyl acetamide, dimethyl formamide, and tetrahyrdofurfuryl alcohol. Other penetration enhancers include amphiphiles such as L-amino acids, anionic surfactants, cationic surfactants, amphoteric surfactants, nonionic surfactants, fatty acids and alcohols. Additional penetration enhancers are disclosed in Remington: The Science and Practice of Pharmacy, 19th Edition (1995) on page 1583. Of course materials such as penetration enhancers can also be premixed with the bioactive material prior to the point of ejection, for example the bioactive material and modifying substance can be present together in one of the container modules 305, 306, and 307.

While each of the container modules 305, 306, and 307 may carry different bioactive materials, it may also be convenient to have each container module 305, 306, and 307 carry the same materials, with controller 200, shown in FIGS. 13 and 14, applying fluid from first container module 305 until empty, followed by fluid from a second container module 306, and so forth. In such a same-fluid embodiment, it would be preferable for controller 200 to indicate to the subject, or an attendant, when fluid is being dispensed from the last delivery system, such as container module 307. This indication may take the form of displaying a message on screen 220, or simply activating an audible alarm or indicator light.

In another embodiment of the present invention, which relates generally to the controlled formation of nanometer-sized particles and/or molecular clusters of substances of interest by a Supercritical Anti-Solvent (SAS) type process as is disclosed in U.S. Ser. No. 10/814,354 filed Mar. 31, 2004 entitled PROCESS FOR THE FORMATION OF PARTICULATE MATERIAL by Rajesh Vinodrai Mehta, et al., U.S. Ser. No. 10/815,026 filed Mar. 31, 2004 entitled PROCESS FOR THE DEPOSITION OF UNIFORM LAYER OF PARTICULATE MATERIAL by Rajesh Vinodrai Mehta, and U.S. Ser. No. 10/815,010 filed Mar. 31, 2004 entitled PROCESS FOR THE SELECTIVE DEPOSITION OF PARTICULATE MATERIAL by Rajesh Vinodrai Mehta, all of which are incorporated by reference herein in their entirety and are explained in detail below.

Now referring to FIG. 1H, like numerals indicate like parts and operations as previously discussed. The SAS process for the formation of particulate material of the desired bioactive material 40 comprises charging a particle formation reservoir 12, the temperature and pressure in which are controlled, with a supercritical fluid, agitating the contents of the particle formation reservoir 12 with a rotary agitator or mixing element 72 comprising an impeller having an impeller surface and an impeller diameter as previously described, creating a relatively highly agitated zone located within a distance of one impeller diameter from the surface of the impeller of the rotary agitator 72, and a bulk mixing zone located at distances greater than one impeller diameter from the surface of the impeller. Introducing into particle formation reservoir 12 the agitated particle at least a first feed stream comprising at least a solvent and the desired substance dissolved therein through a first feed stream introduction port shown as inlet ports 52, 54, and 56 and a second feed stream comprising the supercritical fluid through a second feed stream introduction port also shown as inlet ports 52, 54, and 56, wherein the desired substance is less soluble in the supercritical fluid relative to its solubility in the solvent and the solvent is soluble in the supercritical fluid, and wherein the first and second feed stream introduction ports are located within a distance of one impeller diameter from the surface of the impeller of the rotary agitator such that the first and second feed streams are introduced into the highly agitated zone of the particle formation vessel and the first feed stream is dispersed in the supercritical fluid by action of the rotary agitator, allowing extraction of the solvent into the supercritical fluid, and precipitating particles of the desired substance such as the bioactive material 40 in the particle formation reservoir 12 with a volume-weighted average diameter of less than 100 nanometers.

Using the SAS process described above, it has been found that nanometer sized particles of a desired substance can be prepared by precipitation of the desired substance from a solution upon contact with a supercritical fluid anti-solvent under conditions as described herein. In practicing this invention, feed materials, i.e., the supercritical fluid anti-solvent and the solvent/solute solution are intimately mixed in a particle formation vessel in a zone of highly agitated turbulent flow to precipitate particles of the solute. The particles are then expelled from the highly agitated zone by action of bulk mixing in the particle formation vessel. In practicing the invention, it is generally desirable to introduce the feed streams into the highly agitated mixing zone in opposing directions although they can be introduced in the same direction, if desired. A significant feature of this invention is that precipitated particles of sizes less than 100 nanometers can be produced free of high levels of non-uniform large particles.

In this embodiment of the invention, the process may be performed in an essentially continuous manner by exhausting supercritical fluid, solvent and the desired substance from the particle formation vessel at a rate substantially equal to the rate of addition of such components to the vessel in step, while maintaining temperature and pressure in the vessel at a desired constant level, such that formation of particulate material occurs under essentially steady-state continuous conditions. Such continuous operation is believed to be facilitated by the very fine nature of the precipitated particles, which allows the supercritical fluid, solvent and desired substance to be simply exhausted from the particle formation vessel by passage to an expansion chamber. In such embodiment, passage to the expansion chamber may be through, e.g., a backpressure regulator, a capillary, or a flow distributor. Once passed to the expansion chamber, the particles of the desired substance may be collected without interruption of the precipitation in the agitated particle formation vessel. If desired, supercritical fluid, solvent and desired substance may be exhausted from the particle formation vessel directly into a solution to form a dispersion of the formed particles of the desired substance.

Since the process of the present invention produces fine powder that is comparable to those produced by RESS techniques, RESS-based thin film deposition techniques (including method and apparatus, with minor changes to account for low level of organic solvent present in the supercritical mixture) may also be employed for the particles produced by the present invention.

Very fine particles obtained in accordance with the invention may also be printed, coated, or otherwise deposited upon expansion of the supercritical fluid mixture, similarly as described in the deposition or printing processes of WO 02/45868 A2, U.S. Pat. No. 6,471,327, U.S. Pat. No. 6,692,906, U.S. 2002/0118246 A1, U.S. 2002/0118245 A1, and U.S. 2003/0107614, or as described in pending applications U.S. Ser. No. 10/815,010 and U.S. Ser. No. 10/815,026, the disclosures of which are incorporated by reference herein. Very fine particles obtained in accordance with the invention may further also be printed, coated, or otherwise deposited upon expansion of the supercritical fluid mixture in process similarly as described in copending, commonly assigned U.S. Ser. No. 10/313,549 filed Dec. 6, 2002 entitled SYSTEM FOR PRODUCING PATTERNED DEPOSITION FROM COMPRESSED FLUIDS by Suresh Sunderrajan et al.; U.S. Ser. No. 10/313,587 filed Dec. 6, 2002 entitled METHOD FOR PRODUCING PATTERNED DEPOSITION FROM COMPRESSED FLUID by Ramesh Jagannathan et al.; U.S. Ser. No. 10/460,814 filed Jun. 12, 2003 entitled A METHOD OF MANUFACTURING A COLOR FILTER by Sridhar Sadasivan et al.; U.S. Ser. No. 10/314,379 filed Dec. 6, 2002 entitled SYSTEM FOR PRODUCING PATTERNED DEPOSITION FROM COMPRESSED FLUID IN A DUAL CONTROLLED DEPOSITION CHAMBER by David J. Nelson, et al.; U.S. Ser. No. 10/313,427 filed Dec. 6, 2002 entitled SYSTEM FOR PRODUCING PATTERNED DEPOSITION FROM COMPRESSED FLUID IN A PARTIALLY OPENED DEPOSITION CHAMBER by David J. Nelson et al.; U.S. Ser. No. 10/313,591 filed Dec. 6, 2002 entitled APPARATUS AND METHOD FOR MAKING A LIGHT-EMITTING DISPLAY by Sridhar Sadasivan et al. (supercritical CO₂ based marking system to make organic small molecule and polymeric light emitting diode devices); U.S. Ser. No. 10/224,783 filed Aug. 21, 2002 entitled SOLID STATE LIGHTING USING COMPRESSED FLUID COATING by Ramesh Jagannathan et al.; U.S. Ser. No. 10/300,099 filed Nov. 20, 2002 entitled SOLID STATE LIGHTING USING COMPRESSED FLUID COATINGS by Ramesh Jagannathan et al.; U.S. Ser. No. 0/602,429 filed Jun. 24, 2003 entitled AN APPARATUS AND M ETHOD OF COLOR TUNING A LIGHT-EMITTING DISPLAY by Sridhar Sadasivan et al., U.S. Ser. No. 10/602,134 filed Jun. 24, 2003 entitled A LIGHT EMITTING DISPLAY by Sridhar Sadasivan et al.; U.S. Ser. No. 10/602,430 filed Jun. 24, 2003 entitled AN ARTICLE HAVING MULTIPLE SPECTRAL DEPOSITS by David J. Nelson et al.; U.S. Ser. No. 10/602,840 filed Jun. 24, 2003 entitled AN APPARATUS AND METHOD OF PRODUCING MULTIPLE SPECTRAL DEPOSITS FROM A MIXTURE OF A COMPRESSED FLUID AND A MARKING MATERIAL by David J. Nelson et al.; and U.S. Ser. No. 10/625,426 filed Jul. 23, 2003 entitled AUTHENTICATION METHOD AND APPARATUS FOR USE WITH COMPRESSED FLUID PRINTED SWATCHES by Seshadri Jagannathan et al., the disclosures of which are incorporated by reference herein.

In accordance with various embodiments, the present invention provides technologies that permit functional material deposition of ultra-small particles; that permit high speed, accurate, and precise deposition of a functional material on a receiver; that permits high speed, accurate, and precise patterning of ultra-small features on the receiver; that provide a self-energized, self-cleaning technology capable of controlled functional material deposition in a format that is free from receiver size restrictions; that permits high speed, accurate, and precise patterning of a receiver that can be used to create high resolution patterns on the receiver; that permits high speed, accurate, and precise patterning of a receiver having reduced functional material agglomeration characteristics; that permits high speed, accurate, and precise patterning of a receiver using a mixture of nanometer sized functional material dispersed in dense fluid; that permits high speed, accurate, and precise patterning of a receiver using a mixture of one or more nanometer sized functional materials dispersed in dense fluid and where the nanometer sized functional materials are created by precipitation under steady state conditions; that permits high speed, accurate, and precise patterning of a receiver using a mixture of nanometer sized one or more functional material dispersed in dense fluid and where the nanometer sized functional materials are created as a dispersion in a dense fluid under steady state conditions in a vessel containing a mixing device or devices; that permits high speed, accurate, and precise patterning of a receiver that has improved material deposition capabilities; that provide a more efficient printing method without the previous limitations on the amount of functional material that could be used due to solubility in the compressed fluid; and that permit the use of very small orifice size print head nozzles without the need for filtration by ensuring that the functional material particles are all of a size range not to exceed 2 microns.

The invention has been described with reference to a preferred embodiment, however, it will be appreciated that variations and modifications can be effected by a person of ordinary skill in the art without departing from the scope of the invention.

### PARTS LIST

10 delivery system
11 supercritical fluid source
12 formulation reservoir
12a formulation reservoirs
13 discharge device
14 receiver
15 valve
16 delivery path
17 pressure control mechanism
18 pump
19a, b pressure regulator
20 temperature control mechanism
21 control valve
22 shutter
23 nozzle
24 beam control device
25 spacer-shield
26 nozzle heating module
27 nozzle shield gas module
28 shape
29 shape
30 shape
31 shape
32 shape
32 variable area converging-diverging
33 shape
34 converging section
35 throat section
36 diverging section
37 straight section
38 variable area diverging
38 gas release port
39 solenoid
40 bioactive material
41 compressed liquid
42 formulation
43 beam
44 precipitated bioactive material
45 evaporation
46 particles
47 nozzle opening
48 outlet
49 arrow
50 conveyance mechanism
51 aerodynamic lens
52, 54, 56, inlet ports
53 tubular pipe
57, 61, 63 orifice plates
58 outlet port
59 inlet/outlet ports
60 pump
61 patient
62 delivery path
63 transdermal applicator
64 bioactive material source
65, 66, 67 particles
68 sealed chamber
69 elastomeric seal
70 mixing device
71 inside edge
72 mixing element
73 flexible plastic material
74 power control source
75 skin surface
76 movable piston device
77 programmable microprocessor
78 electrical heating/cooling zones
79 replaceable container module
80 electrical wires
81 keypad
82 water jackets
83 display screen
84 refrigeration coils
85 indicator
86 high-pressure windows
87 memory device
88 control device
89 removable memory
90 microprocessor
91 pulse oximetry sensor
92 transdermal patch
94 upper exposed surface
96 opposing under surface
98 removable protective layer
99 adhesive layer
100 display
105 arrow
110 translation device
120 freeze-dried gelatinous oral material
130 multi-layer time-release material
135a, b, c, d time release layer
140 time release bioactive particle A
141 time release bioactive particle B
142 time release bioactive particle C
143 time release bioactive particle D
144 adhesive carrier material
145 single layer time release material
146 semipermeable layer
147 semipermeable layer
148 semipermeable layer
149 arrow
150 multiple delivery system
151, 152, 153 delivery system
155, 156, 157 bioactive material
160, 161, 162 discharge device
170 web
175 perforations
180 perforations
200 logic and control unit
205 cable
210 modem
215 keyboard
220 display screen
230 button
235 button
300 delivery system
305, 306, 307 container modules
310, 311, 312 discharge device
320, 321, 322 bioactive materials
330 combination
340 point of discharge

What is claimed is:

1. A system for solvent-less delivery of a bioactive material to a receiver, comprising:
a discharge device;
a reservoir which holds a bioactive material; and
a solvent at a supercritical fluid state for delivering said bioactive material through said discharge device to said receiver.

2. A system according to claim 1 wherein said receiver comprising any one of the following:
skin
transdermal patch
freeze-dried gelatinous oral patch
multiyear-layer time release material
absorbent material.

3. A system according to claim 1 wherein the bioactive material is a pharmaceutical composition.

4. A system according to claim 1 wherein the solvent comprises CO2 (carbon dioxide).

5. A system according to claim 1 further comprising a controller which automatically ejects the bioactive material from the discharge device at selected times.

6. A system according to claim 5 wherein the controller is a microprocessor programmed to dispense the bioactive material at predetermined intervals.

7. A system according to claim 1 wherein the reservoir comprises multiple reservoirs.

8. A system according to claim 7 wherein at least one of the reservoirs contains a bioactive material in powder form.

9. A system according to claim 7 wherein at least two of the reservoirs contain different bioactive materials that combine after ejection to produce a bioactive effect.

10. A system according to claim 9 wherein the bioactive material comprises one of the following: proteins and nucleic acids, or liposomes.

11. A system according to claim 1 further comprising an attachment member that selectively retains the discharge device in prolonged contact with the receiver.

12. A system according to claim 111 wherein the attachment member comprises a strap.

13. A system according to claim 111 wherein the attachment member comprises an adhesive.

14. A system according to claim 1 wherein the receiver comprises skin.

15. A system according to claim 1 further comprising an indicator which indicates a degree of depletion of the bioactive material in the reservoir.

16. A system according to claim 1 wherein the receiver comprises skin covering a subject having a measurable parameter, and the system further comprises:
bio-sensor which monitors said parameter of the subject and generates a signal in response thereto; and
a controller which automatically dispenses the bioactive material from the discharge device in response to said signal.

17. A system according to claim 16 wherein the bio-sensor comprises a pulse oximetry device.

18. A system according to claim 16 wherein said parameter comprises pulse rate.

19. A system according to claim 16 wherein said parameter comprises blood oxygenation levels.

20. A system according to claim 16 wherein said bio-sensor communicates said signal to the controller by infrared communication.

21. A system according to claim 16 wherein said bio-sensor communicates said signal to the controller by radio wave communication.

22. A system according to claim 1 further comprising a display which displays information about said bioactive material.

23. A system according to claim 1 further comprising an interface which receives a memory storage device containing dosage information concerning administration of said bioactive material.

24. A system according to claim 1 further comprising a keypad input which receives dosage information concerning administration of said bioactive material.

25. A system according to claim 1 further comprising: a display which displays information about said composition, including various dosages; and a keypad input including scroll keys which when activated cause the display to selectively show said various dosages.

26. A system according to claim 1 further comprising a controller which is programmable.

27. A system according to claim 26 wherein said controller is programmable from a remote computer in communication with said controller.

28. A system according to claim 16 wherein the reservoir comprises two container modules each containing different bioactive materials, the receiver has indicia detectable by said optical sensor indicative of one of said different bioactive substances, and the controller causes said nozzle to eject said one of said different bioactive substances.

29. A system according to claim 1 wherein said receiver comprises transdermal patch having an absorbent material which receives said delivery of said bioactive material.

30. A system according to claim 1 further comprising a pressure control mechanism provided to maintain a desired pressure of said solvent.

31. A system according to claim 30 wherein said pressure control mechanism includes a pump, a valve, and a pressure regulator.

32. A system according to claim 1 further comprising a temperature control mechanism for controlling the temperature of said solvent.

33. A system according to claim 32 wherein said temperature control mechanism is provided at the reservoir.

34. A system according to claim 33 wherein said temperature control mechanism includes a heater.

35. A system according to claim 34 wherein said temperature control mechanism also includes a refrigeration coil.

36. A system according to claim 34 wherein said temperature control mechanism 20 can also include any number for monitoring the temperature of the delivery system.

37. A system according to claim 1 wherein a mixing element is provided in said reservoir.

38. A system according to claim 1 wherein said discharge device comprises a nozzle positioned to provide direct delivery of the solvent toward the receiver 14.

39. A system according to claim 38 wherein said discharge device further comprises a shutter to regulate the flow of the solvent.

40. A system according to claim 39 wherein said discharge also includes a spacer-shield used to collect extraneous particles.

41. A system for solvent-less delivery of a bioactive material to a receiver, comprising: a discharge device, a spacer positioned between the discharge device and the receiver, a reservoir which holds a bioactive material, and a solvent at a supercritical fluid state for delivering said bioactive material through said discharge device to said receiver.

42. A system according to claim 41 wherein the spacer is supported by the discharge device.

43. A system according to claim 41 wherein the spacer is in direct contact with said receiver.

44. A system according to claim 41 wherein the receiver comprises skin, and the spacer comprises a sealing member that selectively substantially seals the spacer against the skin to form a substantially closed chamber between the discharge device and the skin when the spacer is in contact with the skin.

45. A system according to claim 44 wherein said spacer allows escape of gas or vapor resulting from discharge of said solvent.

46. A system according to claim 45 wherein the sealing member is a continuous elastomeric seal.

47. A kit for solvent-less delivery of a bioactive material to a receiver, the kit comprising:
an applicator having a discharge device, a reservoir which holds a bioactive material, and a solvent at a supercritical fluid state for delivering said bioactive material through said discharge device; and
a receiver for receiving said bioactive material.

48. A kit according to claim 47 further comprising instructions for use of said applicator and/or receiver.

49. A kit according to claim 47 further comprising a sensor and/or monitor for monitoring said applicator.

50. A method for solvent-less delivery of a bioactive material to a receiver, comprising:
providing a discharge device, a reservoir which holds a bioactive material, and a solvent at a supercritical fluid state; and
delivering said bioactive material through said discharge device to said receiver.

51. A method according to claim 50 wherein said receiver comprising any one of the following:
skin
transdermal patch
freeze-dried gelatinous oral patch,
multiyear-layer time release material
absorbent material.

52. A method according to claim 50 wherein the bioactive material is a pharmaceutical composition.

53. A method according to claim 50 wherein the solvent comprises CO2 (carbon dioxide).

54. A method according to claim 50 further comprising the step of:
automatically ejecting the bioactive material from the discharge device at selected times.

55. A method according to claim 50 wherein a controller is a microprocessor programmed to dispense the bioactive material at predetermined time intervals.

56. A method according to claim 50 wherein the reservoir comprises multiple reservoirs.

57. A method according to claim 56 wherein at least one of the reservoirs contains a bioactive material in powder form.

58. A method according to claim 56 wherein at least two of the reservoirs contain different bioactive materials that combine after ejection to produce a bioactive effect.

59. A method according to claim 58 wherein the bioactive material comprises one of the following: proteins, nucleic acids, or liposomes.

60. A method according to claim 50 further comprising an attachment member that selectively retains the discharge device in prolonged contact with the receiver.

61. A method according to claim 60 wherein the attachment member comprises a strap.

62. A method according to claim 60, wherein the attachment member comprises an adhesive.

63. A system according to claim 50 wherein the receiver comprises skin.

64. A method according to claim 50 further comprising an indicator which indicates a degree of depletion of the bioactive material in the reservoir.

65. A method according to claim 50 wherein the receiver comprises skin covering a subject having a measurable parameter, and the method further comprises monitoring said measurable parameter of the subject and generating a signal in response thereto.

66. A method according to claim 65 further comprising the step of automatically dispensing the bioactive material from the discharge device in response to said signal.

67. A method according to claim 65 wherein said measurable parameter comprises pulse or blood oxygen level of the subject.

68. A method according to claim 65 wherein said parameter comprises pulse rate.

69. A method according to claim 65 wherein said signal is communicated to a controller by infrared communication.

70. A method according to claim 65 wherein said signal is communicated to a controller by radio wave communication.

71. A method according to claim 50 further comprising displaying information about said bioactive material.

72. A method according to claim 50, further comprising an interface which receives a memory storage device containing dosage information concerning administration of said bioactive material.

73. A method according to claim 50 further comprising a keypad input which receives dosage information concerning administration of said bioactive material.

74. A method according to claim 50 further comprising a display which displays information about said composition, including various dosages, and a keypad input including scroll keys which when activated cause the display to selectively show said various dosages.

75. A method according to claim 50 further comprising the step of programming the automatic delivery of bioactive material.

76. A method according to claim 75 wherein said programming is accomplished from a remote site from said discharge device.

77. A method according to claim 50 further comprising the step of:
detecting when different bioactive material have been provided on to said bioactive material.

78. A method according to claim 50 wherein said receiver comprises dermal patch having an absorbent material which receives said delivery of said bioactive material.

79. A method according to claim 50 further comprising the step of maintaining a desired pressure of said solvent.

80. A method according to claim 50 comprising the step of maintaining a desired temperature of said solvent.

81. A method according to claim 80 further comprising monitoring the temperature of the delivery system.

82. A method according to claim 79 further comprising monitoring the pressure of the delivery system.

83. A method according to claim 50 further comprising the step of mixing solvent in said reservoir.

84. A method according to claim 50 wherein said bioactive material is deposited at a desired depth in said receiver for controlling release of said bioactive material.

85. A method according to claim 50 wherein two or more bio-active materials are placed on said receiver.

86. A method according to claim 85 wherein each of said bioactive material are placed at a different location on said receiver.

87. A method according to claim 85 wherein each of said bio-active material are placed at a different depth on said receiver.

88. A method according to claim 87 where said bio-active material are placed at different depths by controlling the pressure at which they are applied to said receiver.

89. A receiver for applying a bioactive material to a subject, said receiver having a plurality of different bio-active material to be applied to a subject, each located at a different location on said receiver.

90. A receiver for applying a bioactive material to a subject, said receiver having a plurality of different bio-active material to be applied to a subject, each located at a different depth on said receiver.

91. A receiver according to claim 89 wherein said receiver includes absorbent material wherein said bio-material is placed.

92. A receiver according to claim 89 wherein said receiver comprises a plurality of layers each having a different bulk modulus.

93. A receiver according to claim 92 wherein said bulk modulus controls which bio-active material will be placed in said layers.

94. A receiver according to claim 89 wherein said receiver comprises a suppository.
